SEQUENCE LISTING (48 sequences)

SEQ ID NO: 1 - protein, 10 aa, Artificial Sequence
  KRAS WT
KLVVVGAGGV

SEQ ID NO: 2 - protein, 10 aa, Artificial Sequence
  KRASG12V
KLVVVGAVGV

SEQ ID NO: 3 - protein, 10 aa, Artificial Sequence
  KRASG12D
KLVVVGADGV

SEQ ID NO: 4 - protein, 10 aa, Artificial Sequence
  KRASG12C
KLVVVGACGV

SEQ ID NO: 5 - DNA, 318 bp, Homo sapiens
atggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctc

SEQ ID NO: 6 - protein, 106 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYL

SEQ ID NO: 7 - DNA, 327 bp, Homo sapiens
atgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctc

SEQ ID NO: 8 - protein, 109 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYL

SEQ ID NO: 9 - DNA, 327 bp, Homo sapiens
atggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttt

SEQ ID NO: 10 - protein, 109 aa, Homo sapiens
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYF

SEQ ID NO: 11 - DNA, 327 bp, Homo sapiens
atgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctc

SEQ ID NO: 12 - protein, 109 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYL

SEQ ID NO: 13 - DNA, 15 bp, Homo sapiens
agtgttttttccagc

SEQ ID NO: 14 - protein, 5 aa, Homo sapiens
SVFSS

SEQ ID NO: 15 - DNA, 21 bp, Homo sapiens
gtagttacgggtggagaagtg

SEQ ID NO: 16 - protein, 7 aa, Homo sapiens
VVTGGEV

SEQ ID NO: 17 - DNA, 18 bp, Homo sapiens
aacagcgcctcagactac

SEQ ID NO: 18 - protein, 6 aa, Homo sapiens
NSASDY

SEQ ID NO: 19 - DNA, 21 bp, Homo sapiens
attcgttcaaatatggacaaa

SEQ ID NO: 20 - protein, 7 aa, Homo sapiens
IRSNMDK

SEQ ID NO: 21 - DNA, 15 bp, Homo sapiens
ttgaaccacgatgcc

SEQ ID NO: 22 - protein, 5 aa, Homo sapiens
LNHDA

SEQ ID NO: 23 - DNA, 18 bp, Homo sapiens
tcacagatagtaaatgac

SEQ ID NO: 24 - protein, 6 aa, Homo sapiens
SQIVND

SEQ ID NO: 25 - DNA, 15 bp, Homo sapiens
atggggcacagggct

SEQ ID NO: 26 - protein, 5 aa, Homo sapiens
MGHRA

SEQ ID NO: 27 - DNA, 18 bp, Homo sapiens
tacagctatgagaaactc

SEQ ID NO: 28 - protein, 6 aa, Homo sapiens
YSYEKL

SEQ ID NO: 29 - DNA, 45 bp, Homo sapiens
tgtgcaggagagttcgagagtaatgcaggcaacatgctcaccttt

SEQ ID NO: 30 - protein, 15 aa, Homo sapiens
CAGEFESNAGNMLTF

SEQ ID NO: 31 - DNA, 48 bp, Homo sapiens
tgtgccagtagtatttggttgggaccaaaggggcccggggagctgttt

SEQ ID NO: 32 - protein, 16 aa, Homo sapiens
CASSIWLGPKGPGELF

SEQ ID NO: 33 - DNA, 36 bp, Homo sapiens
tgtgcagagaatcgcgacaccgacaagctcatcttt

SEQ ID NO: 34 - protein, 12 aa, Homo sapiens
CAENRDTDKLIF

SEQ ID NO: 35 - DNA, 42 bp, Homo sapiens
tgcgccagcagccttcagatcacaactgatggctacaccttc

SEQ ID NO: 36 - protein, 14 aa, Homo sapiens
CASSLQITTDGYTF

SEQ ID NO: 37 - DNA, 1824 bp, Artificial Sequence
  KTCR1
atgctaggatccatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggagagttcgagagtaatgcaggcaacatgctcacctttggagggggaacaaggttaatggtcaaaccccacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtgctagcggagagggcagaggaagtctgctaacatgcggtgacgtcgaggagaatcctggacctacgcgtatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtatttggttgggaccaaaggggcccggggagctgttttttggagaaggctctaggctgaccgtactggaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccgaattcggcaaataa

SEQ ID NO: 38 - protein, 608 aa, Artificial Sequence
  KTCR1
MLGSMVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGEFESNAGNMLTFGGGTRLMVKPHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSASGSGEGRGSLLTCGDVEENPGPTRMSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIWLGPKGPGELFFGEGSRLTVLDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSEFGR

SEQ ID NO: 39 - DNA, 1824 bp, Artificial Sequence
  KTCR2
atgctaggatccatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaatcgcgacaccgacaagctcatctttgggactgggaccagattacaagtctttccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtgctagcggagagggcagaggaagtctgctaacatgcggtgacgtcgaggagaatcctggacctacgcgtatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtatttggttgggaccaaaggggcccggggagctgttttttggagaaggctctaggctgaccgtactggaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccgaattcggaaggtaa

SEQ ID NO: 40 - protein, 608 aa, Artificial Sequence
  KTCR2
MLGSMAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENRDTDKLIFGTGTRLQVFPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSASGSGEGRGSLLTCGDVEENPGPTRMSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIWLGPKGPGELFFGEGSRLTVLDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSEFGR

SEQ ID NO: 41 - DNA, 1815 bp, Artificial Sequence
  KTCR3
atgctaggatccatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggagagttcgagagtaatgcaggcaacatgctcacctttggagggggaacaaggttaatggtcaaaccccacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtgctagcggagagggcagaggaagtctgctaacatgcggtgacgtcgaggagaatcctggacctacgcgtatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccttcagatcacaactgatggctacaccttcggttcggggaccaggttaaccgttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccgaattcggaaggtaa

SEQ ID NO: 42 - protein, 605 aa, Artificial Sequence
  KTCR3
MLGSMVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGEFESNAGNMLTFGGGTRLMVKPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSASGSGEGRGSLLTCGDVEENPGPTRMGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSLQITTDGYTFGSGTRLTVVDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSEFGA

SEQ ID NO: 43 - DNA, 1815 bp, Artificial Sequence
  PTCR4
atgctaggatccatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaatcgcgacaccgacaagctcatctttgggactgggaccagattacaagtctttccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtgctagcggagagggcagaggaagtctgctaacatgcggtgacgtcgaggagaatcctggacctacgcgtatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccttcagatcacaactgatggctacaccttcggttcggggaccaggttaaccgttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccgaattcggaaggtaa

SEQ ID NO: 44 - protein, 605 aa, Artificial Sequence
  PTCR4
MLGSMAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENRDTDKLIFGTGTRLQVFPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSASGSGEGRGSLLTCGDVEENPGPTRMGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSLQITTDGYTFGSGTRLTVVDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSEFGA

SEQ ID NO: 45 - DNA, 9228 bp, Artificial Sequence
  KTCR-1
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattttaattaaattgccgccatgggatccatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggagagttcgagagtaatgcaggcaacatgctcacctttggagggggaacaaggttaatggtcaaaccccacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtgctagcggagagggcagaggaagtctgctaacatgcggtgacgtcgaggagaatcctggacctacgcgtatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtatttggttgggaccaaaggggcccggggagctgttttttggagaaggctctaggctgaccgtactggaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccgaattcggcaaaggagctactaacttcagcctgctgaagcaggctggagacgtggaggagaaccctggacctggccggcctatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtaaggcgcgccctcgagagatcccccggggtcgactgatcaaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 46 - DNA, 9228 bp, Artificial Sequence
  KTCR-2
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattttaattaaattgccgccatgggatccatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaatcgcgacaccgacaagctcatctttgggactgggaccagattacaagtctttccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtgctagcggagagggcagaggaagtctgctaacatgcggtgacgtcgaggagaatcctggacctacgcgtatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtatttggttgggaccaaaggggcccggggagctgttttttggagaaggctctaggctgaccgtactggaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccgaattcggaagcggagctactaacttcagcctgctgaagcaggctggagacgtggaggagaaccctggacctggccggcctatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtaaggcgcgccctcgagagatcccccggggtcgactgatcaaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 47 - DNA, 9219 bp, Artificial Sequence
  KTCR-3
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattttaattaaattgccgccatgggatccatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggagagttcgagagtaatgcaggcaacatgctcacctttggagggggaacaaggttaatggtcaaaccccacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtgctagcggagagggcagaggaagtctgctaacatgcggtgacgtcgaggagaatcctggacctacgcgtatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccttcagatcacaactgatggctacaccttcggttcggggaccaggttaaccgttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccgaattcggaagcggagctactaacttcagcctgctgaagcaggctggagacgtggaggagaaccctggacctggccggcctatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtaaggcgcgccctcgagagatcccccggggtcgactgatcaaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 48 - DNA, 9183 bp, Artificial Sequence
  PTCR4
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattttaattaaattgccgccatgggatccatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctactttggagggggaacaaggttaatggtcaaaccccacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtgctagcggagagggcagaggaagtctgctaacatgcggtgacgtcgaggagaatcctggacctacgcgtatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccttcagatcacaactgatggctacaccttcggttcggggaccaggttaaccgttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccgaattcggaagcggagctactaacttcagcctgctgaagcaggctggagacgtggaggagaaccctggacctggccggcctatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtaaggcgcgccctcgagagatcccccggggtcgactgatcaaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

(19) United States
(12) Patent Application Publication
(10) Pub. No.: US 2022/0227883 A1
(43) Pub. Date: Jul. 21, 2022

(54) IMMUNOTHERAPY CONSTRUCTS TARGETING KRAS ANTIGENS

(71) Applicant: PROVINCIAL HEALTH SERVICES AUTHORITY, Vancouver (CA)

(72) Inventors: Robert HOLT, Vancouver (CA); Craig RIVE, Vancouver (CA); Simon TURCOTTE, Quebec (CA)

(21) Appl. No.: 17/613,698

(22) PCT Filed: May 26, 2020
(86) PCT No.: PCT/CA2020/050715

§ 371 Date: Nov. 23, 2021

Related U.S. Application Data
(60) Provisional application No. 62/853,102, filed on May 27, 2019.

(51) Int. Cl. C07K 16/32; C07K 14/725; A61P 35/00; G01N 33/574; C12N 5/09; G01N 33/50

(52) U.S. Cl. CPC C07K 16/32; C07K 14/7051; A61P 35/00; G01N 33/57492; C12N 2502/1114; G01N 33/5011; C07K 2317/565; C07K 2317/31; C12N 5/0693

(57) ABSTRACT

An antigen targeting agent is provided. The antigen targeting agent binds to a mutated Kirsten rat sarcoma viral oncogene homolog (KRAS) protein having a missense mutation at position 12 when a peptide incorporating the missense mutation is presented by an HLA-A*02 molecule. The missense mutation at position 12 of the KRAS protein may be G12D, G12V or G12C. The antigen targeting agents can be used diagnostically or for immunotherapy.

### REFERENCE TO RELATED APPLICATIONS

This application claims priority to, and the benefit of, U.S. provisional patent application No. 62/853,102 filed 27 May 2019, which is hereby incorporated herein by reference for all purposes.

### TECHNICAL FIELD

Some embodiments of the present invention relate to peptides, proteins, nucleic acids and cells for use in cancer immunotherapy. Some embodiments of the present invention relate to cancer immunotherapy agents targeting mutant KRAS antigen(s) to stimulate anti-tumour immune responses. Some embodiments of the present invention relate to T-cell receptors targeting tumour-associated KRAS mutant antigen(s). Some embodiments of the present invention relate to compositions and methods for the immunotherapy-based treatment of cancer utilizing antigen targeting agents designed to recognize tumours expressing KRAS antigen(s) presented by HLA-A*02 molecules, including HLA-A*02:01 molecules. Some embodiments of the present invention relate to compositions and methods for the immunotherapy-based treatment of cancer utilizing antigen targeting agents designed to recognize tumours expressing KRAS antigen(s) presented by HLA-A*02 molecules, including HLA-A*02:01 molecules.

### BACKGROUND

There is a general desire for new efficacious and safe cancer treatment options. There is also a general desire for cancer treatment options that are specifically directed to the unique spectrum of mutations that both characterize and have a pathogenic role in the development of a patient's tumour. The existence of mutations specific to each patient's tumours provides the opportunity for a personalized approach to treatment that can be tailored to the genetic makeup of a patient's tumour genotype.

The major histocompatibility complex (“MHC”) is a set of genes that code for cell surface proteins essential for the adaptive immune system. There are two classes of MHC molecules: class I and class II. MHC class I molecules are expressed in all nucleated cells except red blood cells. MHC class I molecules function to mediate cellular immunity, e.g. to flag tumour cells, infected cells, or damaged cells for destruction. MHC Class I molecules are part of a process that presents short peptides (typically 7-12 amino acids in length) to the immune system. The peptides often result from proteolytic cleavage of mainly endogenous, cytosolic or nuclear proteins, defective ribosomal products, and larger peptides expressed by the cell. Under normal conditions, cytotoxic T cells bind to the MHC/peptide complex when the peptide displayed by the MHC molecule is considered as intracellular non-self-derivation, e.g. infected or cancerous cells. If such binding occurs, the binding triggers a cytotoxic response culminating in cell death via apoptosis.

The MHC molecules of humans are designated as human leukocyte-antigens (“HLA”), which can be further divided to subgroups, e.g. HLA-A, HLA-B, and HLA-C. Subgroup HLA-A is one of three major types of human MHC class I cell surface receptors.

HLA alleles are variable in their primary structure. Each HLA allele can be defined by typing at varying levels of resolution. Low resolution typing is a DNA-based typing result at the level of the first field of the classification (formerly the first two digits of the historical four-digit classification system). High resolution typing identifies a set of alleles that encode the same protein sequence for the peptide-binding region of an HLA molecule, and identifies HLA alleles at the resolution of the second field (formerly the second two digits of the historical four-digit classification system). Allelic resolution is DNA-based typing consistent with a single allele. The structure of the classification utilizes a first and second set of digits to reflect the different typing resolutions; e.g. HLA-A*02:01, HLA-A*02:02 and HLA-A*02:04 are members of the A2 serotype. This low resolution typing is the primary factor determining HLA compatibility.

There are several hundred different HLA-A proteins that are known and the frequency of alleles within each serotype varies among racial populations. For example, HLA-A*02:01 is a prevalent allele and it has been reported to be present in about 50% of the US Caucasian population and 17% of the US African American population: Allele Frequencies in Worldwide Populations, as reported online by the Allele Frequency Net Database. Despite the diversity of HLA alleles across global populations, there is some consistency in the HLA binding groove pockets that hold the antigens: Sette A, Sidney J. Nine major HLA class I supertypes account for the vast preponderance of HLA-A and -B polymorphism. Immunogenetics. 1999; 50:201-212. doi: 10.1007/s002510050594.

The KRAS gene (Kirsten rat sarcoma viral oncogene homolog) encodes the K-Ras protein. The K-Ras protein is part of a signaling pathway known as the RAS/MAPK pathway, which relays signals from outside the cell to the cell's nucleus. These signals instruct a cell to grow and divide or to mature and differentiate. When mutated, KRAS has the potential to cause normal cells to become cancerous. Mutated KRAS may be present and expressed in a variety of human cancers, including without limitation pancreatic, colorectal, lung, endometrial, ovarian, and prostate cancers as well as leukemias.

Mutated KRAS proteins are often observed in cancers. Position 12 of the amino acid sequence of KRAS is a mutational hotspot for cancers. For example, it has been reported that KRAS^{G12D} is present in many types of cancer cells, with pancreatic adenocarcinoma, colon adenocarcinoma, lung adenocarcinoma, colorectal adenocarcinoma, and rectal adenocarcinoma having the greatest prevalence: Cancer Discovery. 2017; 7(8):818-831. Dataset Version 6. Similarly, KRAS^{G12V} has been reported to be present in about 3% of the American Association for Cancer Research's Genomics Evidence Neoplasia Information Exchange (GENIE) cases, with pancreatic adenocarcinoma, lung adenocarcinoma, colon adenocarcinoma, colorectal adenocarcinoma, and rectal adenocarcinoma having the greatest prevalence: Cancer Discovery. 2017; 7(8):818-831. Dataset Version 6. Another example is the KRAS^{G12C} mutation that has been reported to be present in about 2% of the GENIE cases, with lung adenocarcinoma, colon adenocarcinoma, non-small cell lung carcinoma, colorectal adenocarcinoma, and adenocarcinoma of unknown primary having the greatest prevalence: Cancer Discovery. 2017; 7(8):818-831. Dataset Version 6.

Focusing on pancreatic ductal adenocarcinoma (PDAC) as an example, which is the fourth leading cause of cancer-related deaths in North America, most PDAC tumors harbour KRAS^{G12D} and KRAS^{G12V} mutations. In particular, KRAS^{G12D} and KRAS^{G12V} are found in approximately 50%, and 30%, of PDAC patients, respectively: Jones, S. et al. “Core signaling pathways in human pancreatic cancers revealed by global genomic analyses.” Science 321, 1801-6 (2008). Such mutations lock the K-Ras protein in an activated state, and have proven to be largely undruggable (i.e. small molecules that inhibit the activity of such mutant versions of K-Ras have proven elusive).

Additionally, KRAS mutations, including mutations at amino acid 12 of KRAS, including KRAS^{G12D}, KRAS^{G12V} and KRAS^{G12C} mutations, are driver mutations that occur early in carcinogenesis and are retained by tumor cells due to oncogene addiction: Weinstein, I. B. Cancer. Addiction to oncogenes—the Achilles heal of cancer. Science 297, 63-4 (2002). As such, the KRAS^{G12} mutational antigens, including KRAS^{G12D}, KRAS^{G12V} and KRAS^{G12C} are an attractive target for cancer screening and/or therapy.

Some KRAS antigens/peptides are able to bind to MHC class I molecules to thereby form a MHC/peptide complex. The MHC/peptide complex can be recognized by a suitable antigen targeting moiety of a cytotoxic cell, e.g. a T-cell receptor of a cytotoxic T-cell, to stimulate an anti-tumour immune response.

In addition to T-cell receptors that can be used to conduct T-cell therapy using cytotoxic T-cells (e.g. via TCR therapy), other types of antigen targeting receptors such as chimeric antigen receptors (e.g. via CAR-T therapy) and the like can be used in the diagnosis, prophylaxis and/or treatment of cancer using cellular immunotherapy using cytotoxic cells tumour-infiltrating lymphocytes (TIL) such as CD8⁺ or CD4⁺ T-cells, natural killer (NK) cells, and so on. Such cells and antigen targeting receptors can be administered to patients via adoptive cell therapy, as allogenic cells, and so on.

Immunogenic agents that can target cells expressing the mutated K-Ras protein and assist in selectively killing such cells have potential efficacy in the diagnosis, treatment and/or prophylaxis of cancer.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides an antigen binding receptor having an antigen binding site configured to specifically bind to a KRAS^{G12D/V/C} peptide-MHC class I molecule complex. In some embodiments, the KRAS^{G12D/V/C} peptide has the amino acid sequence of any one of SEQ ID NO:2, SEQ ID NO:3 or SEQ ID NO:4. In some embodiments, the MHC class I molecule is HLA-A*02. In some embodiments, the MHC class I molecule is HLA-A*02:01.

One aspect of the invention provides an antigen targeting agent that binds to a mutated Kirsten rat sarcoma viral oncogene homolog (KRAS) protein having a missense mutation at position 12 when a peptide incorporating the missense mutation is presented by an HLA-A*02 molecule.

In some embodiments, the missense mutation at position 12 of the KRAS protein is G12D, G12V or G12C.

In some embodiments, the HLA-A*02 molecule is HLA-A*02:01.

In some embodiments, the antigen targeting agent has first and second chains, each one of the first and second chains having first, second and third complementarity determining regions (CDRs). The third CDR of the first chain has the amino acid sequence of SEQ ID NO:30 or SEQ ID NO:34, and the third CDR of the second chain has the amino acid sequence of SEQ ID NO:32 or SEQ ID NO:36.

In some embodiments, the antigen targeting agent has a first chain having the amino acid sequence of TRAV27*01 (SEQ ID NO:6) or the amino acid sequence of TRAV13-2*01 (SEQ ID NO:10).

In some embodiments, the antigen targeting agent has a second chain having the amino acid sequence of TRBV 19*01 (SEQ ID NO:8) or the amino acid sequence of TRBV 04-1*01 (SEQ ID NO:12).

In some embodiments, the antigen targeting agent has a first chain having a first CDR having the amino acid sequence of SEQ ID NO:14 or SEQ ID NO:18.

In some embodiments, the antigen targeting agent has a first chain having a second CDR having the amino acid sequence of SEQ ID NO:16 or SEQ ID NO:20.

In some embodiments, the antigen targeting agent has a second chain having a first CDR having the amino acid sequence of SEQ ID NO:22 or SEQ ID NO:26.

In some embodiments, the antigen targeting agent has a second chain having a second CDR having the amino acid sequence of SEQ ID NO:24 or SEQ ID NO:28.

In some embodiments, the antigen targeting agent has (i) a first chain having as its first, second and third CDRs SEQ ID NO:14, SEQ ID NO:16 and SEQ ID NO:30, respectively, and a second chain having as its first, second and third CDRs SEQ ID NO:22, SEQ ID NO:26 and SEQ ID NO:32, respectively, (ii) a first chain having as its first, second and third CDRs SEQ ID NO:18, SEQ ID NO:20 and SEQ ID NO:34, respectively, and a second chain having as its first, second and third CDRs SEQ ID NO:22, SEQ ID NO:24 and SEQ ID NO:32, respectively; (iii) a first chain having as its first, second and third CDRs SEQ ID NO:14, SEQ ID NO:16, and SEQ ID NO:30, respectively, and a second chain having as its first, second and third CDRs SEQ ID NO:26, SEQ ID NO:28 and SEQ ID NO:36, respectively; or (iv) a first chain having as its first, second and third CDRs SEQ ID NO:18, SEQ ID NO:20 and SEQ ID NO:34, respectively, and a second chain having as its first, second and third CDRs SEQ ID NO:26, SEQ ID NO:28 and SEQ ID NO:36, respectively.

In some embodiments, the antigen targeting agent targets KRAS^{G12V} mutations and the CDR3 of the first chain has the amino acid sequence of SEQ ID NO:30 and the CDR3 of the second chain has the amino acid sequence of SEQ ID NO:32.

In some embodiments, the antigen targeting agent targets KRAS^{G12D} mutations and the CDR3 of the first chain has the amino acid sequence of SEQ ID NO:34 and the CDR3 of the second chain has the amino acid sequence of SEQ ID NO:32.

In some embodiments, the antigen targeting agent targets KRAS^{G12D} mutations and the CDR3 of the first chain has the amino acid sequence of SEQ ID NO:30 and the CDR3 of the second chain has the amino acid sequence of SEQ ID NO:36.

In some embodiments, the first and second chains of the antigen targeting agent form a single polypeptide or the first and second chains of the antigen targeting agent form two separate polypeptides.

In some embodiments, the first and second chains of the antigen targeting agent are configured to be expressed as a single polypeptide with a suitable sequence interposing the first and second chains so that the first and second chains are cleaved into or expressed as two separate polypeptides in vivo. The, suitable sequence can be a T2A, P2A, E2A, F2A or IRES sequence.

In some embodiments, the antigen targeting agent is a T-cell receptor (TCR). In some such embodiments, the first chain is an alpha-chain of the TCR, and the second chain is a beta-chain of the TCR. In other such embodiments, the first chain is a gamma-chain of the TCR, and the second chain is a delta-chain of the TCR.

In some embodiments, the antigen targeting agent is a chimeric antigen receptor (CAR), and the three complementarity determining regions of each of the first and second chains are configured to be expressed as a single polypeptide together with a co-stimulatory domain.

In some embodiments, the antigen targeting agent is a bi-specific antibody, the bi-specific antibody having a first domain having the antigen binding site that binds to the KRAS protein having a missense mutation at position 12 when a peptide incorporating the missense mutation is presented by an HLA-A*02 molecule, and a second domain comprising an antigen binding site configured to bind to cytotoxic cells. In some such embodiments, the second domain of the bi-specific antibody binds CD3.

Another aspect of the invention provides a T-cell receptor having the amino acid sequence of any one of SEQ ID NOs:38, 40, 42 or 44.

Another aspect of the invention provides an isolated nucleic acid molecule having a DNA sequence encoding an antigen targeting agent or T-cell receptor as described herein. In some embodiments, the isolated nucleic acid molecule has the nucleotide sequence of any one of SEQ ID NOs:37, 39, 41, 43, 45, 46, 47 or 48.

Another aspect of the invention provides a cytotoxic cell capable of expressing an antigen binding agent or an engineered T-cell receptor as described herein.

Another aspect of the invention provides a method of producing a cytotoxic cell capable of expressing an antigen targeting receptor to target KRAS peptides having a missense mutation at position 12 as presented by HLA-A*02 molecules. The method includes isolating cytotoxic cells from a source and genetically engineering the immune cells using a nucleotide vector as described herein. The cells can be used to conduct autologous or allogenic adoptive cell therapy.

In some embodiments, the method involves sequencing a sample from the subject to verify the presence of KRAS having a missense mutation at position 12 and/or HLA typing to verify that the subject has an HLA-A*02 allele. The HLA typing may be used to verify that the subject has an HLA-A*02:01 allele.

Another aspect provides a method of detection of cancer in a mammal. The method involves contacting a sample comprising cells with an antigen targeting agent as described herein, if the cells express KRAS^{G12X} antigens, the antigen targeting agent binds to the KRAS^{G12X} antigens, thereby forming a complex; and the presence of the complex is detected, wherein the presence of the complex is indicative of cancer in the mammal.

Another aspect provides a method of detection of cancer in a mammal. The method involves obtaining a sample from the subject; co-culturing cells from the sample with cytotoxic cells capable of binding to KRAS^{G12X} peptides as displayed by HLA-A*02 molecules; and evaluating an indicator of cytotoxic activity. The presence of the indicator of cytotoxic activity or an increase in the level of the indicator of cytotoxic activity indicates cancer involving a mutation at position 12 of the KRAS protein.

Another aspect of the present invention provides a method to treat a patient with cancer with an engineered TCR that recognizes a KRAS epitope.

In some embodiments, the engineered TCR has alpha and beta chains having any pairwise combination of the variable regions and/or the CDRs having the amino acid sequences of SEQ ID NOs: 38, 40, 42 and 44.

In some embodiments, murine constant gene segments are incorporated into the TCR alpha and beta chains of the present invention, in place of human constant gene segments, in order to limit mispairing of the engineered TCR alpha and beta chains with the T cell's endogenous TCR alpha and beta chains.

Another aspect of the invention provides related nucleic acids, recombinant vectors, host cells, populations of cells and pharmaceutical compositions relating to the TCRs, polypeptides and proteins of the invention.

Methods of identification of patients responsive to treatment by the present invention based on tumour KRAS mutation screening, HLA typing or other methods of patient screening are also provided by the invention.

Methods of detecting the presence of cancer in a mammal and methods of treating or preventing cancer in a mammal are further provided by the invention.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 shows a block diagram outlining a modified mini-line T-cell expansion protocol for the purpose of screening donor T-cell repertoires for antigen-specific T-cells.

FIG. 2 shows an example of Gamma interferon (IFNγ) ELISpot analysis of mini-line expanded CD8⁺ T-cell polyclonal pools.

FIG. 3 shows an example of the single cell sorting flow cytometry gating protocol.

FIGS. 4A-4J show an example of tetramer analysis of T-cell clones.

FIG. 5 shows an example of assessment by IFNγ ELISpot of T-cell clone target specificity.

FIG. 6 shows a schematic representation showing an example embodiment of a complete TCR recombinant construct (“KTCR-1”) for reconstitution.

FIG. 7 shows a schematic representation showing an example embodiment of a complete TCR recombinant construct (“KTCR-2”) for reconstitution.

FIG. 8 shows a schematic representation showing an example embodiment of a complete TCR recombinant construct (“KTCR-3”) for reconstitution.

FIGS. 9A, 9B, 9C and 10A-10D show the results of KTCR-1, KTCR-2, and KTCR-3 lentivirus titration over HeLa cells in order to determine an optimal amount of the lentivirus required in transfection.

FIG. 11 shows the results of sorting KTCR-X transduced CD8+ T cells showing those cells positive for the mStrawberry reporter gene.

FIG. 12 shows raw ELISpot data which was analysed using Graphpad—Prism 8 (v. 8.0.0).

FIGS. 13A, 13B, 13C, 13D, 13E and 13F show sample flow cytometry data analysis of K562-A:02:01 pulsed with KRAS^{G12D} peptide and co-cultured with KTCR-2 cells and control lymphocytes.

FIG. 14 shows the raw data histogram plots of FSV780 live/dead stained cells.

FIG. 15 shows the analysis of the raw data shown of FIG. 14.

FIG. 16 shows an annotated version of the nucleotide sequence of KTCR-1 with mouse constant regions (SEQ ID NO:37).

FIG. 17 shows an annotated version of the amino acid sequence (SEQ ID NO:38) translated from the nucleotide sequence of KTCR-1.

FIG. 18 shows an annotated version of the nucleotide sequence of KTCR-2 with mouse constant regions (SEQ ID NO:39).

FIG. 19 shows an annotated version of the amino acid sequence (SEQ ID NO:40) translated from the nucleotide sequence of KTCR-2.

FIG. 20 shows an annotated version of the nucleotide sequence of KTCR-3 with mouse constant regions (SEQ ID NO:41).

FIG. 21 shows an annotated version of the amino acid sequence (SEQ ID NO:42) translated from the nucleotide sequence of KTCR-3.

FIG. 22 shows a multiple sequence alignment of the amino acid sequences of KTCR-1, KTCR-2, KTCR-3 and the predicted sequence of PTCR-4 (SEQ ID NOs:38, 40, 42 and 44). Complementarity determining regions (CDRs) in each sequence are underlined.

FIG. 23 shows Gamma Interferon (IFN-γ) ELISpot analysis of KRAS^{G12V} and KRAS^{G12D} specific, HLA-A*02:01-restricted reconstituted T-cell receptors (rTCR).

FIG. 24 shows tetramer staining of KRAS^{G12V} and KRAS^{G12D} specific, HLA-A*02:01-restricted TCRs.

FIGS. 25A and 25B show testing results of HLA-A*02:01-restricted KRAS^{G12V} specific TCR reconstituted T cells in vivo.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein, the terms “CD8⁺ T-cells” and “TCD8⁺” refer to CD8-positive T-cells. CD8-positive T-cells are able recognize and destroy cells flagged by MHC class I molecules and this ability is known as MHC class I-restriction. CD8-positive T-cells include cytotoxic T-cells (CTLs). Similarly, “CD4⁺ T-cells” refers to CD4-positive T-cells.

As used herein, the term “antigen” refers to molecules that can induce an immune response. For example, an antigen may be one that is recognisable by cytotoxic T-cells to stimulate an anti-tumour immune response.

As used herein, the term “epitope” refers to the part of an antigen that can stimulate an immune response. For example, an epitope may be a peptide that is bound to a MHC class I molecule to thereby form a MHC/peptide complex. The MHC/peptide complex can be selectively recognized by a suitable T-cell receptor of a cytotoxic T-cell to stimulate an anti-tumour immune response.

As used herein, the term “DNA” refers to deoxyribonucleic acid. The information stored in DNA is coded as a sequence made up generally of four chemical bases: adenine (A), guanine (G), cytosine (C) and thymine (T). Other bases and chemically modified bases exist as well and are encompassed within certain embodiments. As used herein, reference to a DNA sequence includes both single and double stranded DNA. A specific sequence refers to (i) a single stranded DNA of such sequence, (ii) a double stranded DNA comprising a single stranded DNA of such sequence and its complement, and (iii) the complement of such sequence.

As used herein, the term “fragment” means a portion of a larger whole. In the context of a DNA coding sequence, a fragment means a portion of the DNA sequence that is less than the complete coding region. However, the expression product of the fragment may retain substantially the same biological function as the expression product of the complete coding sequence.

As used herein, the term “peptide” means a series of amino acid residues, connected to each other by peptide bonds between the alpha-amino and carbonyl groups of the adjacent amino acid. A peptide may be immunogenic, meaning that the peptide is capable of inducing an immune response, e.g. a T-cell response.

As used herein, the term “isolated” means that a material is separated/removed from its original environment. For example, HLA-A*02:01:KRAS^{G12D&V}-reactive CD8⁺ T cells removed from their natural environment, e.g. blood, are isolated. HLA-A*02:01:KRAS^{G12D&V}-reactive CD8⁺ T cells present their natural environment within a pancreatic cancer patient are not isolated.

As used herein, the term “purified” does not mean absolute purity. Instead, it can include preparations that undergo a purification process, e.g. highly purified preparations and partially purified preparations having a purity of at least about 50%, at least about 60%, at least about 70%, at least about 80%, at least about 90%, at least about 95%, at least about 97%, at least about 98%, or at least about 99% pure.

As used herein, the term “T-cell response” means the proliferation and activation of effector T-cells. For example, T-cell response of MHC class I restricted cytotoxic T-cells may include lysis of target cells, secretion of cytokines, and secretion of effector molecules (e.g. perforins and granzymes).

As used herein, the term “variant” means in the context of proteins, one or two or more of the amino acid residues are replaced with other amino acid residues, while the variant retains substantially the same biological function as the unaltered protein.

The terms “treat”, “treating” and “treatment” refer to an approach for obtaining desired clinical results. Desired clinical results can include, but are not limited to, reduction or alleviation of at least one symptom of a disease. For example, treatment can be diminishment of at least one symptom of disease, diminishment of extent of disease, stabilization of disease state, prevention of spread of disease, delay or slowing of disease progression, palliation of disease, diminishment of disease reoccurrence, remission of disease, prolonging survival with disease, or complete eradication of disease.

The terms “cancer cell” and “tumor cell” refer to cells, the growth and division of which can be typically characterized as unregulated. Cancer cells can be of any origin, including benign and malignant cancers, metastatic and non-metastatic cancers, and primary and secondary cancers.

As used herein, the term “KRAS^{G12X}” refers to KRAS missense mutants at KRAS codon position 12. As used herein, the term “KRAS^{G12D&V}” refers to KRAS^{G12D} and KRAS^{G12V} mutant KRAS, i.e. KRAS having a missense mutation at position 12 wherein the wild type glycine residue is mutated to an aspartic acid residue or a valine, respectively. “KRAS^{G12C}” refers to KRAS in which the wild type glycine residue at position 12 is mutated to a cysteine residue.

In one embodiment, the inventors have discovered an antigen targeting receptor targeting KRAS^{G12X} antigens/mutants that can be used to stimulate anti-tumour immune responses. In some embodiments, the antigen targeting receptor is a T-cell receptor. The T-cell receptor is engineered to recognize and bind to KRAS^{G12X} antigens/mutant peptides that are presented by MHC class I molecules of the subclass HLA-A*02:01. Because many cancer cells express KRAS^{G12X} antigens/mutants and because HLA-A*02:01 is a highly prevalent HLA-A subtype, the novel antigen targeting receptor of some embodiments can be used for cancer screening, treatment and prevention in a large segment of the patient population. For example, cytotoxic cells such as CD8⁺ T cells may be engineered to express the novel antigen targeting receptors, e.g. as T-cell receptors (TCRs) or chimeric antigen receptors (CARs). When the TCRs or CARs recognize and bind to KRAS^{G12X} antigens expressed on tumour cells and presented by HLA-A*02:01, CD8+ T cells are activated and can kill the tumour cells, e.g. through lysis of the tumour cells, secretion of cytokines, and/or secretion of effector molecules (e.g. perforins and granzymes).

### Antigen Targeting Agents

Some embodiments of the present invention relate to antigen targeting agents, including antigen targeting receptors. These antigen targeting agents are configured to target KRAS^{G12X} antigens presented by HLA-A*02 molecules to stimulate anti-tumour immune responses, for example by positioning cytotoxic cells such as T-cells adjacent tumour cells to promote killing of the tumour cells by the cytotoxic cells. In some embodiments, these antigen targeting agents are configured to target KRAS^{G12X} antigens presented by HLA-A*02:01 molecules.

In some embodiments, these antigen targeting agents are specific for KRAS^{G12X} antigens as displayed by HLA-A*02 molecules, meaning that the agents can specifically bind to and immunologically recognize KRAS^{G12X} antigens with high avidity. For example, an antigen targeting agent may be considered to have antigenic specificity for KRAS^{G12X} antigens if T cells expressing a TCR incorporating the antigen targeting agent secrete at least twice as much IFNγ upon co-culture with HLA-A*02:01 positive antigen presenting cells (APC) (e.g. K562b cells modified to express HLA-A*02:01) pulsed with the KRAS^{G12X} peptide having a relevant target mutation at position 12 of KRAS as compared to the amount of IFNγ expressed by a negative control. IFNγ secretion may be measured by methods known in the art such as, for example, enzyme-linked immunosorbent assay (ELISA).

In some embodiments, the targeted KRAS^{G12X} antigens are KRAS^{G12D/V/C} antigens. Wild type KRAS (KRAS^{WT}) contains a ten amino acid fragment having the sequence KLVVVGAGGV (SEQ ID NO:1). In some embodiments, the targeted KRAS^{G12DN} antigens have the amino acid sequences set forth in SEQ ID NO:2 (KLVVVGAVGV, a peptide corresponding KRAS having a missense mutation at position 12 of G12V, referred to herein as KRAS^{G12V}) and SEQ ID NO:3 (KLVVVGADGV, a peptide corresponding to KRAS having a missense mutation at position 12 of G12D, referred to herein as KRAS^{G12D}). In some embodiments, the targeted KRASGI2X antigens are KRAS^{G12C} antigens having the amino acid sequence set forth in SEQ ID NO:4 (KLVVVGACGV, a peptide corresponding to KRAS having a missense mutation at position 12 of G12C).

In some embodiments, the targeted KRAS^{G12X} antigens are variants of SEQ ID NOs:2-4 or other peptides incorporating a missense mutation at position 12 of KRAS that vary in length, e.g. that contain one, two, three, four or five additional amino acids from the KRAS protein at the N-terminus and/or at the C-terminus of the peptide, and/or which contain one, two or three fewer amino acids from the KRAS protein at the N-terminus and/or one or two fewer amino acids at the C-terminus of the peptide. In some embodiments, the targeted antigens have additional amino acids at the N-terminal and/or C-terminal end of the peptide, e.g. one, two, three, four or five additional amino acids at the N-terminus of the peptide, and/or one, two, three, four or five additional amino acids at the C-terminus of the peptide. In some embodiments, the targeted antigens have fewer amino acids at the N-terminal and/or C-terminal end of the peptide e.g. with one, two or three amino acids removed from the KRAS protein at the N-terminus and/or one or two amino acids removed at the C-terminus of the peptide. In some embodiments, the targeted KRAS^{G12X} antigens are 8-mer, 9-mer, 10-mer, 11-mer, 12-mer, 13-mer, 14-mer, 15-mer or 16-mer peptides incorporating the missense mutation at position 12 of KRAS.

In some embodiments, the antigen targeting agents have an antigen binding site that is specific for KRAS^{G12X} antigens presented at the cell surface by HLA-A*02 molecules. In some embodiments, the HLA-A*02 molecules are HLA-A*02:01 molecules.

In some embodiments, the antigen targeting agents target cytotoxic cells to tumour cells. For example, in some embodiments, the antigen targeting agent is a T-cell receptor (TCR) that targets a T-cell incorporating the construct to tumour cells expressing the target missense mutation at position 12 of KRAS. In some embodiments, the antigen targeting agent is a chimeric antigen receptor (CAR) that targets a cytotoxic cell such as a T-cell to tumour cells expressing the target missense mutation at position 12 of KRAS. In some embodiments, the antigen targeting agent is an agent such as a bi-specific antibody that has a first antigen-binding domain that binds to a target KRAS^{G12X} antigen as presented by HLA-A*02 molecules to target the agent to tumour cells and a second antigen-binding domain that targets cytotoxic cells, for example that binds to CD3 to target T-cells to the tumour cells.

Any type of immunotherapy agent that can be used to target cytotoxic cells to tumour cells can be used in various embodiments. In some embodiments, bispecific antibodies that bind to both a KRAS^{G12X} antigen presented at the cell surface by HLA-A*02 molecules and a factor such as CD3 that can be used to target cytotoxic cells such as T-cells to the tumour cells bound by the bispecific antibody can be used. In some embodiments, an antigen targeting receptor that can be used to conduct cellular immunotherapy can be used. In some embodiments, the antigen targeting receptor is a T-cell receptor (TCR). In some embodiments, the antigen targeting receptor is a chimeric antigen receptor (CAR). In some embodiments, the antigen targeting receptor is a modified form of TCR-CAR construct with a single chain antigen-binding domain of a TCR fused to the signaling domain of a CAR molecule.

In some embodiments, the antigen targeting agent is a TCR. The TCR has (i) a first chain having first, second and third complementarity-determining regions (CDR1, CDR2, and CDR3) and (ii) a second chain having first, second and third complementarity-determining regions (CDR1, CDR2, and CDR3). In some embodiments, the first and second chains of the TCR are the alpha chain and beta chain, respectively, of a TCR. In some embodiments, the first and second chains of the TCR are the gamma chain and delta chain, respectively, of a TCR. Without being bound by theory, the third complementarity-determining regions (CDR3) are believed to play an important role in KRAS^{G12X} antigen binding and specificity whereas the first and second complementarity-determining regions (CDR1 and CDR2) are believed to play a role in binding to the MHC Class I backbone (e.g. to the HLA-A*02 molecules). TCR sequences, like antibody sequences, are generated by somatic VDJ recombination and are highly stochastic.

The design and structure of synthetic TCRs generally is known in the art. In some embodiments, each of the first and second chains of the synthetic TCRs has one or more of the following domains: a hinge domain, a transmembrane domain, and an intracellular T-cell signalling domain. In some embodiments, the intracellular domains of the TCR do not signal directly, but rather form complexes with other molecules such as CD3 subunits that facilitate signalling.

In some embodiments in which the antigen targeting agent is a T-cell receptor, the antigen targeting agent is expressed from a nucleotide construct capable of expressing both chains of the TCR as a single polypeptide. In some embodiments, the single polypeptide has a linker peptide linking the first and second chains of the T-cell receptor. The linker peptide may facilitate the expression of a recombinant TCR in a host cell.

In some embodiments, the single polypeptide incorporating both the first and second chains of the synthetic TCR includes a cleavage sequence interposed between the first and second chains of the TCR, so that the first and second chains will be expressed as a single polypeptide and then cleaved into two separate polypeptides in vivo. In some embodiments, the nucleic acid encoding the polypeptide that forms the TCR includes a skipping sequence or a sequence allowing initiation of translation at a site other than the 5′ end of an mRNA molecule, or any other sequence that allows two distinct polypeptides to be translated from a single mRNA, interposed between the nucleic acid encoding the first and second chains of the TCR. Any suitable sequence may be used for this purpose between the first and second chains of the TCR, for example a T2A, P2A, E2A, F2A, or IRES sequence, or the like.

The order of the first and second chains of the synthetic TCRs in the polynucleotide sequence encoding the TCR and in the resulting polypeptide is interchangeable (i.e in some embodiments, the first chain is provided at the 5′ end of the polynucleotide sequence/the N-terminal direction of the polypeptide, while in other embodiments the second chain is provided at the 5′ end of the polynucleotide sequence/the C-terminal direction of the polypeptide). In some embodiments, the variable domains of the α chain (V_{α}) and the β chain (V_{β}) comprise any pairwise combination of the variable regions and/or the CDRs having the amino acid sequences of SEQ ID NOs: 38, 40, 42 and 44.

In some embodiments, the constant domains of the first and second chains, e.g. the alpha chain (C_{α}) and the beta chain (C_{β}) comprise human constant gene segments. In other embodiments, human constant gene segments are replaced with constant gene segments from a different organism, e.g. with murine constant gene segments. An advantage of such replacement is to limit mispairing of the engineered TCR chains, e.g. alpha and beta chains, with the T cell's endogenous T-cell receptor chains, e.g. alpha and beta chains.

In some embodiments, the constant domains of the first and second chains are further modified in any suitable manner to enhance and/or regulate the interaction therebetween. For example residues of the transmembrane domains of each of the first and second chains that are positioned adjacent to one another in vivo may be changed to cysteine residues, to encourage the formation of additional disulfide bonds between the engineered first and second chains (while such disulfide bonds would not form with endogenous T-cell receptor chains).

In some embodiments, instead of using TCR constant domains to form a dimer between the first and second chains of the TCR, the synthetic TCRs are provided with any other suitable protein domain that supports dimerization of the two chains, for example a leucine zipper domain.

In some embodiments, the CDR3 of the alpha chain has the amino acid sequence set forth in SEQ ID NO:30 or the amino acid sequence set forth in SEQ ID NO:34. In some embodiments, the CDR3 of the beta chain has the amino acid sequence set forth in SEQ ID NO:32 or the amino acid sequence set forth in SEQ ID NO:36.

The first and second complementarity-determining regions (CDR1 and CDR2) can have any amino acid sequences as long as they are configured to engage with KRAS^{G12X}peptides presented by HLA-A*02 molecules, including HLA-A*02:01 molecules. For example, in some embodiments, the CDR1 of the alpha chain has the amino acid sequence set forth in SEQ ID NO:14 or the amino acid sequence set forth in SEQ ID NO:18. In some embodiments, the CDR2 of the alpha chain has the amino acid sequence set forth in SEQ ID NO:16 or the amino acid sequence set forth in SEQ ID NO:20.

In some embodiments, the CDR1 of the beta chain has the amino acid sequence set forth in SEQ ID NO:22 or the amino acid sequence set forth in SEQ ID NO:26. In some embodiments, the CDR2 of the beta chain has the amino acid sequence set forth in SEQ ID NO:24 or the amino acid sequence set forth in SEQ ID NO:28.

In some embodiments, the TCR has (i) an alpha chain having first, second and third complementarity-determining regions (CDR1, CDR2, and CDR3) having the amino acid sequences set forth in SEQ ID NO:14, SEQ ID NO:16 and SEQ ID NO:30, respectively; and (ii) a beta chain having first, second and third complementarity-determining regions (CDR1, CDR2, and CDR3) having the amino acid sequences set forth in SEQ ID NO:22, SEQ ID NO:24 and SEQ ID NO:32.

In other embodiments, the TCR has (i) an alpha chain having first, second and third complementarity-determining regions (CDR1, CDR2, and CDR3) having the amino acid sequences set forth in SEQ ID NO:18, SEQ ID NO:20 and SEQ ID NO:34, respectively; and (ii) a beta chain having first, second and third complementarity-determining regions (CDR1, CDR2, and CDR3) having the amino acid sequences set forth in SEQ ID NO:22, SEQ ID NO:24 and SEQ ID NO:32.

In other embodiments, the TCR has (i) an alpha chain having first, second and third complementarity-determining regions (CDR1, CDR2, and CDR3) having the amino acid sequences set forth in SEQ ID NO:14, SEQ ID NO:16 and SEQ ID NO:30, respectively; and (ii) a beta chain having first, second and third complementarity-determining regions (CDR1, CDR2, and CDR3) having the amino acid sequences set forth in SEQ ID NO:26, SEQ ID NO:28 and SEQ ID NO:36.

In other embodiments, the TCR has (i) an alpha chain having first, second and third complementarity-determining regions (CDR1, CDR2, and CDR3) having the amino acid sequences set forth in SEQ ID NO:18, SEQ ID NO:20 and SEQ ID NO:34, respectively; and (ii) a beta chain having first, second and third complementarity-determining regions (CDR1, CDR2, and CDR3) having the amino acid sequences set forth in SEQ ID NO:26, SEQ ID NO:28 and SEQ ID NO:36.

In some embodiments, the antigen targeting agent has first and second chains, which may be formed as a single polypeptide or as two separate polypeptides, each of the first and second chains having CDRs, the CDRs independently having any combination of the sequences of the CDRs set forth in Table 4.

In some embodiments, the engineered antigen targeting receptor has any one of the amino acid sequences set forth in SEQ ID NO:38, SEQ ID NO:40, SEQ ID NO:42 or SEQ ID NO:44.

In some embodiments, the engineered antigen targeting receptor is transduced into the T-cell using a viral vector having the nucleotide sequence of the plasmid of any one of SEQ ID NOs:45, 46, 47 or 48.

In some embodiments, the alpha chain and the beta chain of the TCRs are interchangeable, i.e. can be expressed in any desired order from a suitable expression vector. The variable domains of the α chain (V_{α}) and the β chain (V_{β}) comprise any pairwise combination of the variable regions and/or the CDRs of the sequences of SEQ ID NOs: 38, 40, 42 and 44.

Suitable variations on such constructs can be made by those skilled in the art, for example the antigen-binding domains of a T-cell receptor can be inserted into a CAR construct in place of the typical scFv fragment together so that the single-chain antigen-binding domain interacts with the signaling domain of the CAR construct to cause the desired cytotoxic activity towards cancer cells.

In some embodiments, the antigen targeting agent is a chimeric antigen receptor (CAR). In such embodiments, the CAR is structured to provide a single-chain antigen binding domain equivalent to the TCR binding domain described above having the first and second chains (e.g. alpha and beta chains) of the TCR (each having three complementarity determining regions, which may be any of the complementarity determining regions described above for the TCR construct) joined together as a single polypeptide and linked together to a single hinge region, transmembrane domain and signalling domain, as well as a suitable co-stimulatory domain, (e.g. CD27, CD28, 4-1BB, ICOS, OX40, MYD88, IL1R1, CD70, or the like), as well as any other domains intended to enhance the characteristics of the CAR construct.

In some embodiments, the antigen targeting agent is a bispecific antibody, wherein the bispecific antibody has a first antigen-binding domain that binds to a factor such as CD3 that can be used to recruit T-cells and a second antigen-binding domain that binds to a KRAS^{G12X} mutant peptide displayed by an HLA-A*02 molecule, including an HLA-A*02:01 molecule. In one example embodiment, the second domain of the bispecific antibody has as a single polypeptide the first and second chains (e.g. alpha and beta chains) of a TCR as described herein (each having three complementarity determining regions, which may be any of the complementarity determining regions described herein for the TCR construct) to provide the second antigen-binding domain.

Some embodiments of the present invention relate to nucleic acids, recombinant vectors, host cells, populations of cells and pharmaceutical compositions relating to, incorporating or encoding the TCRs, polypeptides and proteins described above.

### Conduct of Immunotherapy Using Antigen Targeting Agents

In some embodiments, the antigen targeting agents described above, such as TCRs or CARs, are introduced into cytotoxic cells in any suitable manner, to provide a cytotoxic cell that specifically targets and kills cells expressing a form of KRAS that is mutated at position 12 as presented by HLA-A*02 molecules such as HLA-A*02:01 molecules. In some embodiments, the mutant KRAS is KRAS^{G12D}, KRAS^{G12V} or KRAS^{G12C}.

Examples of cytotoxic cells that can be used in various embodiments include tumour infiltrating lymphocytes (TILs), including CD8⁺ T-cells, CD4⁺ T-cells, natural killer (NK) cells, and the like. Any cell that can be engineered to carry out cellular immunotherapy can be used in alternative embodiments.

The antigen targeting construct can be introduced into the cytotoxic cell using any suitable technique now known or later developed. In some embodiments, the antigen targeting construct is introduced into the cytotoxic cell using plasmid or RNA transfection, transduction by viral vectors, direct editing via programmable nucleases (e.g. CRISPR systems (clustered regularly interspaced short palindromic repeats), TALENs (transcription activator-like effector nucleases), zinc finger nucleases, and so on as known to those skilled in the art. In some embodiments, the antigen targeting construct is introduced into the cytotoxic cell by transduction with a suitable a vector, e.g. lentiviral or retroviral vectors, adenoviruses, adeno-associated virus (AAV), transposons, and the like. In some embodiments, the antigen targeting construct is introduced into the cytotoxic cell using a transposon system or electroporation.

In some embodiments, the desired antigen targeting receptor is used to generate engineered cytotoxic cells using autologous adoptive cell therapy. That is, the cytotoxic cells are harvested from a mammalian subject, genetically engineered to express the antigen targeting receptor, expanded ex vivo, and then the expanded cells are introduced back into the subject to treat the cancer associated with cells expressing the mutant form of KRAS having a missense mutation at position 12, e.g. KRAS^{G12D}, KRAS^{G12V} or KRAS^{G12C}. In some embodiments, the mammalian subject is a human.

In some embodiments, the desired antigen targeting receptor is used to generate engineered cytotoxic cells using universal adoptive cell therapy using allogenic cells. In universal adoptive cell therapy, a bank of cells from an allogenic donor are genetically modified to express the desired antigen targeting receptor, such as a TCR or CAR as described herein. The modified allogenic cells are then introduced into a patient to treat a cancer associated with cells expressing a mutant form of KRAS, e.g. KRAS^{G12D}, KRAS^{G12V} or KRAS^{G12C}. The patient can be a mammalian subject, e.g. a human.

In some embodiments, the desired antigen targeting receptor is introduced into a mammalian subject, e.g. a human, using systemic gene therapy. For example, a replication incompetent viral vector containing a nucleotide sequence for expressing the antigen targeting receptor is directly infused into a patient to directly transduce T-cells in situ to treat a cancer associated with cells expressing a mutant form of KRAS, e.g. KRAS^{G12D}, KRAS^{G12V} or KRAS^{G12C}.

In some embodiments rather than engineering cytotoxic cells, the desired antigen targeting receptor is converted into a suitable soluble immunotherapy agent, for example a bi-specific antibody such as a bi-specific T-cell engager (BITE®), that can be directly administered to a mammalian subject. In such an embodiment, the portions of the first and second chains that form the antigen-binding region (each containing first, second and third CDRs) are combined together as a single polypeptide that targets tumour cells expressing mutant KRAS as displayed by HLA-A*02 molecules, including HLA-A*02:01 molecules, and are expressed as a fusion protein together with a second antigen binding domain, e.g. an scFv that binds to T-cells e.g. via the CD3 receptor. The resulting fusion protein is purified and administered to the subject in any suitable manner to direct cytotoxic T-cells to the tumour cells.

Methods of administration of the cellular immunotherapy agents and immunotherapy agents described herein are known in the art, and may include, for example, intravenous or subcutaneous injection.

In some embodiments, the likelihood that a mammalian subject will benefit from therapy using an antigen targeting agent described herein are conducted prior to commencing such therapy. A sample from the subject is evaluated to determine if the subject may have potentially cancerous cells that have a missense mutation at position 12 of KRAS. For example, a sample of a tumour from the patient may be subjected to DNA sequencing or appropriate analytical techniques to determine the presence of such a mutation. The mammalian subject is also subjected to HLA typing, to determine if the subject has an HLA-A*02 allele and/or which HLA-A allele the subject has. If the subject has both potentially cancerous cells that have a missense mutation at position 12 of KRAS and an HLA-A*02 allele, including in some embodiments an HLA-A*02:01 allele, then the subject is a potential candidate for immunotherapy using the antigen targeting agents described herein.

In one specific example embodiment, engineered TCRs as described herein are incorporated into CD8+ T cells. When the T-cell receptor recognizes and bind to KRAS^{G12D/V/C} antigens presented by HLA-A*02 molecules (e.g. HLA*02:01 molecules) on tumour cells, the CD8+ T cells are activated and can bind to the tumour cells and initiate a cytotoxic response to kill the tumour cells, e.g. through lysis of the tumour cells, secretion of cytokines, and/or secretion of effector molecules (e.g. perforins and granzymes).

In one specific example embodiment, the T-cell receptors are synthesized and reconstituted in CD8+ T cells using lentiviral transduction. The lentiviral transduction uses a nucleotide vector encoding a receptor comprising an antigen binding domain capable of binding to KRAS^{G12D/V/C} antigens presented by HLA-A*02 molecules (e.g. HLA-A*02:01 molecules). In some embodiments, the nucleotide vector includes nucleotides having a DNA sequence of any one of SEQ ID NOs:37, 39, 41 or 43.

In some embodiments, immune cells capable of binding to KRAS^{G12D/V/C} antigens and initiating a cytotoxic response are made. They are made by first isolating the immune cells from a source of cells and genetically engineering the immune cells to express a receptor comprising an antigen binding domain capable of binding to KRAS^{G12D/V/C} antigens as displayed at the cell surface by HLA-A*02 molecules. In some aspects, the genetic engineering can be carried out using a lentiviral vector. The engineered immune cells can be introduced into the body of a patient having an HLA-A*02 subtype and suffering from cancer or another disorder involving expression of KRAS^{G12D/V/C} to treat the cancer or the disorder. In some embodiments, the patient has an HLA-A*02:01 subtype.

The engineered CD8+ T cells may be used to treat a patient with cancer and/or to screen for cancer. Focusing on an example illustrating the treatment aspect, because KRAS^{G12DN} is a prevalent and mutation in patients suffering from pancreatic ductal adenocarcinoma (PDAC), the engineered CD8+ T cells may be particularly effective as an immunotherapeutic for such pancreatic cancers. Additionally, KRAS^{G12X} is the most common cancer hotspot mutation and HLA-A*02:01 is a prevalent HLA allele, so a large patient population stands to benefit, and such benefit extends beyond PDAC to other cancer types with these common mutations such as lung and colorectal adenocarcinoma.

In some embodiments, the engineered immunotherapy receptors targeting KRAS^{G12X} antigens are used in a patient having an HLA-A*02 subtype in a method for treating or preventing cancer. For example, the method may be chimeric antigen receptor (CAR) T-cell therapy or T-cell receptor (TCR) T-cell therapy.

In some embodiments, methods of identification of patients responsive to treatment by the present invention based on tumour KRAS mutation screening, HLA typing or other methods of patient screening are also provided.

### Screening Using Antigen Targeting Agents

In some embodiments, the antigen targeting agents targeting KRAS^{G12X} antigens displayed at the cell surface by HLA-A*02 molecules are used to detect the presence of tumour cells in a sample such as a patient biopsy. In some such embodiments, detection is made by conducting an assay to evaluate the ability of cytotoxic cells expressing the antigen targeting receptor to kill tumour cells in a tumour cell culture derived from the sample, or by evaluating the expression of molecules that indicate activation of cytotoxic cells, such as interferon-gamma, when such cells are co-cultured with tumour cells (e.g. using ELISpot).

In some embodiments, the antigen targeting agents targeting KRAS^{G12X} antigens are used to detect the presence of tumour cells in a sample such as blood, for example by detecting such antigens displayed on episomes, i.e. membrane fragments that have been shown to be present in blood. In some embodiments, an in vitro assay using the synthetic TCRs, for example using the TCR as a labelled soluble reagent or expressed in a cell with a reporter system as described below can detect the presence of such antigens displayed on episomes.

In some embodiments, the engineered antigen targeting receptors are used for detecting the presence of cancer in a mammal. For example, the engineered antigen targeting receptors (their related polypeptides, proteins, nucleic acids, recombinant expression vectors, or engineered cells) may be brought into contact with a sample having one or more cells or episomes. If the cells express KRAS^{G12X} antigens that are displayed by HLA-A*02 molecules, the engineered antigen targeting receptors will bind to the KRAS^{G12X} antigens and thereby form a complex. The detection of the complex is indicative of the presence of potentially cancerous or pre-cancerous cells.

The detection of the complex may take place through any number of ways known in the art. In some embodiments, the engineered antigen targeting agents (and/or their related polypeptides, proteins, nucleic acids, recombinant expression vectors, or engineered cells) may be labeled with a detectable and/or visual label, e.g. a radioisotope or a fluorophore.

In some embodiments, the engineered antigen targeting receptors are reconstituted in immortalized T-cell lines (e.g. Jurkat cells) to support in vitro high throughput screening assays, for example for use in research and development and/or drug discovery. By way of non-limiting example, in some embodiments, the antigen targeting receptors are reconstituted in a soluble tetrameric form of an αβ TCR, i.e. a TCR multimer, and used diagnostically, e.g. to visualize cells exposed to infectious agents or cellular transformation and/or therapeutically, e.g. for the delivery of drugs to compromised cells, for example as described by Low et al. PloS One, 7(12), e51397, 2012. In some other embodiments, the engineered antigen targeting receptors are reconstituted in reporter cells derived from the T cell lymphoma line Jurkat as reported by Rydzek et al., Molecular Therapy, 27(2), 287-299, 2019.

### EXAMPLES

Certain embodiments are further described with reference to the following examples, which are intended to be illustrative and not limiting in nature.

### Example 1—Isolation of HLA-A*02:01:KRAS^{G12D&V} Reactive CD8⁺ T Cells

Clonally pure populations of HLA-A*02:01:KRAS^{G12D&V}-reactive CD8⁺ T cells were isolated from peripheral blood mononuclear cells (PBMC) from a pancreatic cancer patient. Their target specificity to KRAS^{G12D&V} antigens displayed by HLA-A*02:01 molecules was verified.

The TCR alpha and beta chains from HLA-A*02:01:KRAS^{G12D&V}-reactive CD8⁺ T cell clones were sequenced, resynthesized and reconstituted as recombinant TCRs in healthy donor CD8+ T cells using lentiviral transduction.

The screening protocol to identify HLA-A*02:01:KRAS^{G12D&V}-reactive CD8⁺ T cells was a modified “mini-line” culture method. The protocol is described in e.g. Wick et al., Clinical Cancer Research. 2014 Mar. 1; 20(5):1125-34. doi: 10.1158/1078-0432.CCR-13-2147. PMID: 24323902; Martin et al., A library-based screening method identifies neoantigen-reactive T cells in peripheral blood prior to relapse of ovarian cancer. Oncolmmunology. 2017 Sep. 21; 7(1):e1371895. doi: 10.1080/2162402X.2017.1371895. eCollection 2017. PMID: 29296522. Each of the foregoing publications is incorporated by reference herein.

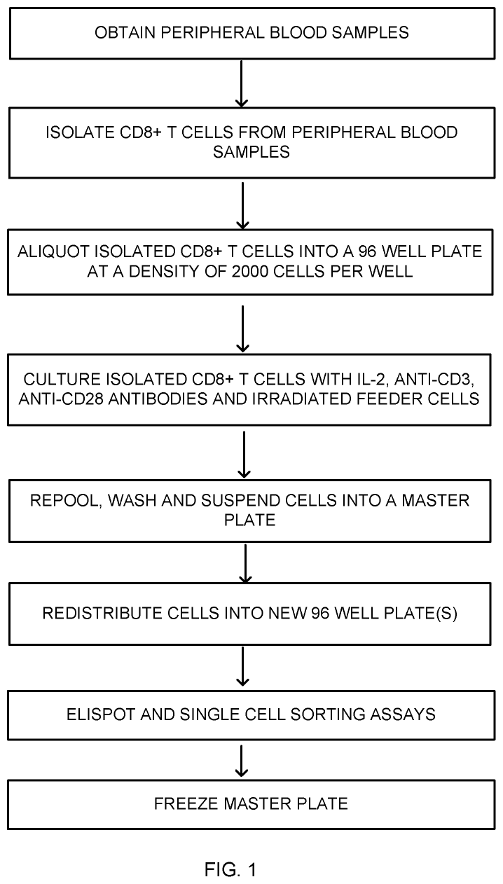

The modified mini-line T-cell expansion protocol is schematically shown in FIG. 1. Peripheral blood samples from Pancreatic Ductal Adenocarcinoma (PDAC) patients were obtained from the BC Pancreas Centre. Peripheral blood mononuclear cells (PBMC) were purified from whole blood, and CD8⁺ T cells were isolated from PBMC using the CD8⁺ T cell isolation kit following the recommended protocol outlined by the manufacturer (Miltenyi Biotec, Bergisch Gladbach. Germany) and were aliquoted into a 96 well plate with U shaped wells (Thermo Fisher, CA. USA) at a density of 2000 cells per well. Cells were then cultured in RPMI-1640 supplemented media (Thermo Fisher, CA. USA) with additional rIL-2 (300U/mL) (PreproTech, NJ. USA), anti-CD3 (Clone OKT3, BioLegend San Diego, Calif., USA) and anti-CD28 antibodies (Clone CD28.2, BioLegend San Diego, Calif. USA) at a final concentration of 1 μg/mL and irradiated feeder cells from a control PBMC source at a ratio of 1:1000 (T-cell:feeder). Day 5 and every 2nd day thereafter the cultures were split and RPMI-1640 supplemented media with additional rIL-2 (final concentration 300U/mL) was added until the end of the expansion on day 14. Day 14, cells were re-pooled into a master plate, washed, resuspended in RPMI-1640 supplemented media with only a small amount of rIL-2 (10U/mL), and incubated for 4 days before performing ELISpot and single cell sorting assays.

### Example 2—Screening for Reactivity to KRAS^{G12DN} Peptides

The panel of polyclonal T-cell pools was then screened for reactivity to KRAS^{G12DN} peptides in the context of HLA-A*02:01 using IFN-γ (interferon gamma) ELISPOT assays (MabTech).

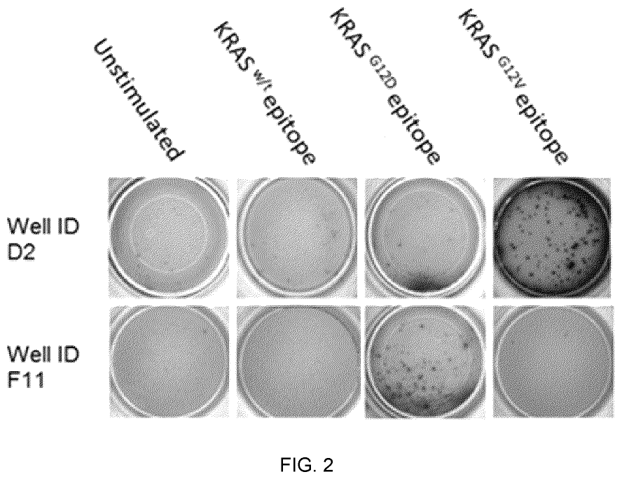

As shown in FIG. 2, several polyclonal T-cell pools showed an antigen-specific IFN-γ response by ELISPOT and these were subsequently re-stimulated with HLA-A*02:01 positive antigen presenting cells (APC) (K562b cells modified to express HLA-A*02:01) pulsed with the KRAS^{G12D} peptide having an amino acid sequence as set forth in SEQ ID NO:3 and KRAS^{G12V} peptide having an amino acid sequence as set forth in SEQ ID NO:2. Post-expansion pools were exposed to antigen presenting cells (APCs) pulsed with KRAS^{G12D/G12V} predicted HLA-A*02:01-restricted epitopes (Genscript, NJ. USA) for 24-28 hours in vitro (APC/T-cell ratio 1:5). ELISpot plate development was performed following the standard ELISpot protocol outlined by the manufacturer and supplier of the ELISpot detection antibodies and materials (MABTECH, Stockholm. Sweden).

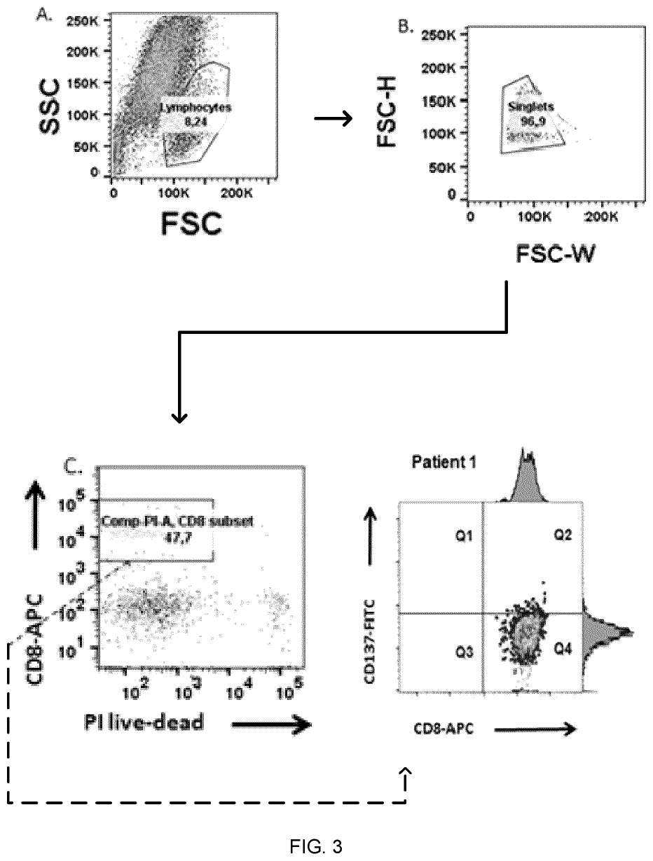

As shown in FIG. 3, reactive T-cells were single-cell sorted by Fluorescence Activated Cell Sorting (FACS) based on detection of de novo expression of the transient activation marker 4-1BB (CD137). The ELISpot positive live polyclonal T-cells from Patient 1 were sorted into single cells based on the expression of CD8, the transient, antigen-induced activation marker, CD137 using a propium iodide (PI)-live/dead stain (BD Biosciences, NJ. USA) and the fluorochrome labelled antibodies CD8-APC and CD137-FITC (eBiosciences, Thermo Fisher, CA. USA) (Q2, Quadrant 2) after 24 hours in co-culture with APCs pulsed with KRAS^{G12D/G12V} predicted HLA-A*02:01-restricted epitopes.

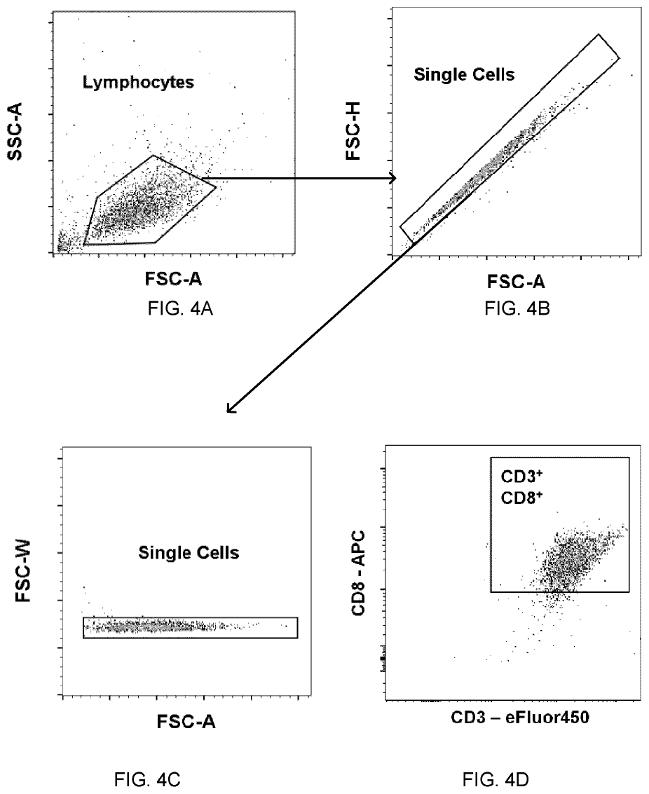

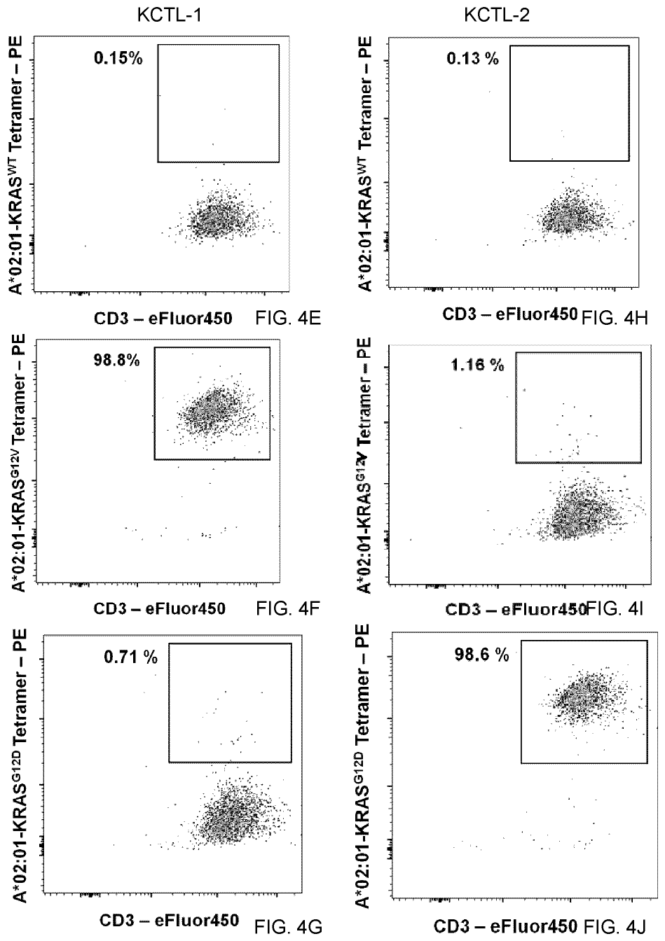

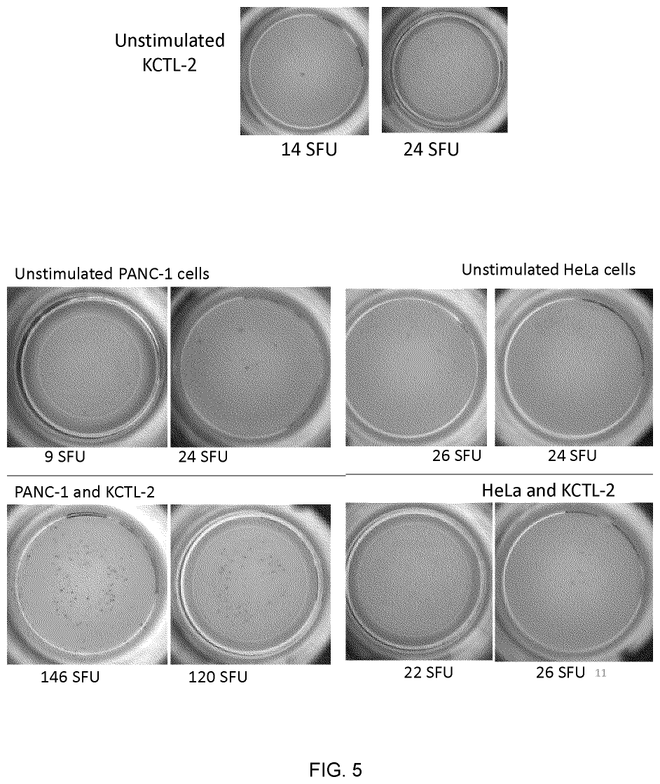

Single sorted T-cells were expanded in cRPMI media supplemented with IL-2 (200U/mL) and an excess of allogeneic irradiated PBMC feeders. To explore the function and specificity of anti-KRAS^{G12X} monoclonal T-cell populations, some of the candidate T-cell clones were assessed by HLA-A*02:01-KRAS^{G12X} tetramer staining (as shown in FIGS. 4A-4J), and/or by IFN-γ ELISPOT for reactivity to cell lines carrying both the HLA-A*02:01 allele and the relevant KRAS^{G12X} mutation (as shown in FIG. 5 and Table 1).

With reference to FIGS. 4A-4J, tetramers were designed based the HLA-A*02:01 presentation of the KRAS^{wild type}, KRAS^{G12V}, and KRAS^{G12D} predicted epitopes and labeled with the PE fluorochrome (NIH Tetramer facility, GA. USA). Isolation of single cells is shown in FIGS. 4A, 4B and 4C. With reference to FIGS. 4D to 4J, CD3-eFluor 450 is shown along the X axis. KCTL-1 KRAS^{G12V} HLA-A*02:01-restricted peptide-specific T-cell clone stained positive for CD3 and CD8 (FIG. 4D), and the A*02:01-KRAS^{G12V} tetramer (FIG. 4F), but negative for both the A*02:01-KRAS^{G12D} (FIG. 4G) and A*02:01-KRAS^{wild type} (FIG. 4E). KCTL-2 KRAS^{G12D} HLA-A*02:01-restricted peptide-specific T-cell clone stained positive for CD3 and CD8 (FIG. 4D), and the A*02:01-KRAS^{G12D} (FIG. 4J) but negative for both the A*02:01-KRAS^{G12V} (FIG. 4I) and A*02:01-KRAS^{wild type} (FIG. 4H). Fluorochrome labeled antibody anti-CD3-eFluor 450 (eBiosciences, Thermo Fisher, CA. USA) and CD8-APC (eBiosciences, Thermo Fisher, CA. USA).

With reference to FIG. 5, the KRAS^{G12D} HLA-A*02:01-restricted peptide-specific T-cell clone (“KCTL-2”) were activated when co-cultured with PANC-1 and HeLa cells in RPMI-1640 supplemented media (Thermo Fisher, CA. USA). The media also contained 10U/mL of rIL-2 (PreproTech, NJ. USA). The co-culture of 25,000 PANC-1 cells and 25,000 KCTL-2, showed an increase in gamma interferon (IFNγ) spot forming units (SFU) when compared to both PANC-1 and KCTL-2 alone. Furthermore, when the KCTL-2 was co-cultured with the non-HLA-A*02:01/non-KRAS^{G12D} HeLa cell line, under the same conditions, no notable variation was detected in the SFUs. Presented are examples of the raw ELISpot well images for KCTL-2, tabulated results from all wells are listed in Table 1, ELISpot plate development was performed following the standard ELISpot protocol outlined by the manufacturer and supplier of the ELISpot antibodies and materials (MABTECH, Stockholm, Sweden) except for an additional wash step to account for the adherent nature of PANC-1 and HeLa cells.

Table 1 below summarizes the IFNγ ELISpot data as interpreted from the raw data, sample results of which are presented in FIG. 5. Table 1 includes the SFU of IFNγ per 2.5×10⁴ KCTL-2 cells normalised against controls to account for non-specific/background spots. Table 1 also includes mean, standard deviation (SD), and number of replicates (N). A significant difference between the SFU of IFNγ in KCTL-2 and PANC-1 co-cultures when compared to KCTL-2 and HeLa co-cultures was determined using a two-tailed T test with p values shown below.

TABLE 1
Summary of example IFNγ ELISpot data.

| | KCTL-2 (SFU of | | | | | | | | | p value (two- |
|---|---|---|---|---|---|---|---|---|---|---|
| | IFNγ/2.5 × 10⁴ cell input) | | | | | | Mean | SD | N | tailed T test) |
| PANC-1 | 97 | 129 | 103 | 100 | 41 | 146 | 86 | 34.9 | 6 | ** 0.0002 |
| HeLa | 8 | 6 | 11 | 2 | 0 | 0 | 4 | 4.7 | 6 | |

The above data show cytolytic activity of the candidate TCRs is target specific. That is, there is selectivity towards the cognate neoantigen (G12D or G12V) used to isolate each TCR, and no specific recognition of the wild-type version of the KRAS 5-14aa epitope.

### Example 3—Prediction for Binding of Different HLA-A*02 Subtypes to KRAS^{G12D/V/C} Peptides

Binding predictions for various HLA-A*02 alleles to KRAS^{G12D/V/C} peptides were carried out using NetMHCpan v3.0 (Nielsen, M., & Andreatta, M. (2016), Genome Medicine, 8(1), 33). An IC₅₀ threshold of 500 nM was used to distinguish binding (IC₅₀<500 nM) from non-binding peptides (IC₅₀>500 nM). The HLA-A*02 alleles that are predicted to bind to KRAS^{G12D/V/C} peptides are shown in Table 2.

About 154 distinct HLA-A*02 alleles were predicted to be able to bind to KRAS^{G12D}. About 184 distinct HLA-A*02 alleles were predicted to be able to bind to KRAS^{G12V}. About 180 distinct HLA-A*02 alleles were predicted to be able to bind to KRAS^{G12C}.

TABLE 2
HLA-A*02 alleles predicted to bind to various KRAS^{G12X}
peptides and predicted binding affinity (IC₅₀, nM).

| G12V | | G12C | | G12D | |
|---|---|---|---|---|---|
| Allele | IC₅₀ | Allele | IC₅₀ | Allele | IC₅₀ |
| HLA-A02: 253 | 37.5 | HLA-A02: 03 | 32.1 | HLA-A02: 253 | 43.3 |
| HLA-A02: 03 | 37.5 | HLA-A02: 253 | 32.1 | HLA-A02: 03 | 43.3 |
| HLA-A02: 264 | 37.5 | HLA-A02: 230 | 32.1 | HLA-A02: 264 | 43.3 |
| HLA-A02: 258 | 37.5 | HLA-A02: 258 | 32.1 | HLA-A02: 258 | 43.3 |
| HLA-A02: 230 | 37.5 | HLA-A02: 264 | 32.1 | HLA-A02: 230 | 43.3 |
| HLA-A02: 69 | 37.6 | HLA-A02: 11 | 36.1 | HLA-A02: 69 | 67.2 |
| HLA-A02: 11 | 37.6 | HLA-A02: 69 | 36.1 | HLA-A02: 11 | 67.2 |
| HLA-A02: 128 | 58.3 | HLA-A02: 128 | 59.2 | HLA-A02: 104 | 78 |
| HLA-A02: 104 | 65.6 | HLA-A02: 22 | 59.3 | HLA-A02: 22 | 78 |
| HLA-A02: 22 | 65.6 | HLA-A02: 104 | 59.3 | HLA-A02: 50 | 83.9 |
| HLA-A02: 50 | 71.5 | HLA-A02: 50 | 64 | HLA-A02: 128 | 107.2 |
| HLA-A02: 26 | 79 | HLA-A02: 26 | 80.4 | HLA-A02: 26 | 112.5 |
| HLA-A02: 171 | 79 | HLA-A02: 171 | 80.4 | HLA-A02: 171 | 112.5 |
| HLA-A02: 141 | 87.5 | HLA-A02: 99 | 88.8 | HLA-A02: 99 | 116.6 |
| HLA-A02: 99 | 90.9 | HLA-A02: 13 | 102.2 | HLA-A02: 102 | 139.2 |
| HLA-A02: 13 | 109.7 | HLA-A02: 02 | 108.8 | HLA-A02: 155 | 139.2 |
| HLA-A02: 90 | 111.3 | HLA-A02: 63 | 108.8 | HLA-A02: 63 | 139.2 |
| HLA-A02: 158 | 111.3 | HLA-A02: 102 | 108.8 | HLA-A02: 02 | 139.2 |
| HLA-A02: 131 | 112.1 | HLA-A02: 115 | 108.8 | HLA-A02: 186 | 139.2 |
| HLA-A02: 16 | 112.1 | HLA-A02: 209 | 108.8 | HLA-A02: 115 | 139.2 |
| HLA-A02: 102 | 123.9 | HLA-A02: 155 | 108.8 | HLA-A02: 209 | 139.2 |
| HLA-A02: 155 | 123.9 | HLA-A02: 186 | 108.8 | HLA-A02: 47 | 163 |
| HLA-A02: 63 | 123.9 | HLA-A02: 141 | 113.3 | HLA-A02: 13 | 167.5 |
| HLA-A02: 02 | 123.9 | HLA-A02: 90 | 119.8 | HLA-A02: 141 | 191.7 |
| HLA-A02: 186 | 123.9 | HLA-A02: 47 | 122.1 | HLA-A02: 90 | 220.4 |
| HLA-A02: 115 | 123.9 | HLA-A02: 158 | 128.5 | HLA-A02: 148 | 226.3 |
| HLA-A02: 209 | 123.9 | HLA-A02: 16 | 149.6 | HLA-A02: 158 | 233.2 |
| HLA-A02: 47 | 138.8 | HLA-A02: 131 | 149.6 | HLA-A02: 131 | 237.4 |
| HLA-A02: 29 | 142.1 | HLA-A02: 148 | 163.8 | HLA-A02: 16 | 237.4 |
| HLA-A02: 263 | 142.2 | HLA-A02: 263 | 176.8 | HLA-A02: 263 | 306.1 |
| HLA-A02: 116 | 152.8 | HLA-A02: 29 | 178.1 | HLA-A02: 116 | 315.6 |
| HLA-A02: 241 | 162.7 | HLA-A02: 12 | 178.9 | HLA-A02: 29 | 320.5 |
| HLA-A02: 71 | 162.7 | HLA-A02: 116 | 185.1 | HLA-A02: 35 | 341.9 |
| HLA-A02: 59 | 162.7 | HLA-A02: 27 | 189.4 | HLA-A02: 38 | 348.1 |
| HLA-A02: 40 | 162.7 | HLA-A02: 105 | 196.9 | HLA-A02: 105 | 354 |
| HLA-A02: 166 | 162.7 | HLA-A02: 73 | 203.4 | HLA-A02: 12 | 356.3 |
| HLA-A02: 238 | 162.7 | HLA-A02: 245 | 203.4 | HLA-A02: 245 | 357.4 |
| HLA-A02: 176 | 162.7 | HLA-A02: 01 | 203.6 | HLA-A02: 73 | 357.4 |
| HLA-A02: 75 | 162.7 | HLA-A02: 09 | 203.6 | HLA-A02: 241 | 360.8 |
| HLA-A02: 30 | 162.7 | HLA-A02: 31 | 203.6 | HLA-A02: 71 | 360.8 |
| HLA-A02: 174 | 162.7 | HLA-A02: 40 | 203.6 | HLA-A02: 59 | 360.8 |
| HLA-A02: 266 | 162.7 | HLA-A02: 24 | 203.6 | HLA-A02: 40 | 360.8 |
| HLA-A02: 187 | 162.7 | HLA-A02: 25 | 203.6 | HLA-A02: 166 | 360.8 |
| HLA-A02: 85 | 162.7 | HLA-A02: 30 | 203.6 | HLA-A02: 238 | 360.8 |
| HLA-A02: 165 | 162.7 | HLA-A02: 59 | 203.6 | HLA-A02: 176 | 360.8 |
| HLA-A02: 160 | 162.7 | HLA-A02: 66 | 203.6 | HLA-A02: 75 | 360.8 |
| HLA-A02: 183 | 162.7 | HLA-A02: 67 | 203.6 | HLA-A02: 30 | 360.8 |
| HLA-A02: 189 | 162.7 | HLA-A02: 68 | 203.6 | HLA-A02: 174 | 360.8 |
| HLA-A02: 138 | 162.7 | HLA-A02: 70 | 203.6 | HLA-A02: 266 | 360.8 |
| HLA-A02: 228 | 162.7 | HLA-A02: 71 | 203.6 | HLA-A02: 187 | 360.8 |
| HLA-A02: 260 | 162.7 | HLA-A02: 74 | 203.6 | HLA-A02: 85 | 360.8 |
| HLA-A02: 107 | 162.7 | HLA-A02: 75 | 203.6 | HLA-A02: 165 | 360.8 |
| HLA-A02: 215 | 162.7 | HLA-A02: 77 | 203.6 | HLA-A02: 160 | 360.8 |
| HLA-A02: 182 | 162.7 | HLA-A02: 85 | 203.6 | HLA-A02: 183 | 360.8 |
| HLA-A02: 09 | 162.7 | HLA-A02: 86 | 203.6 | HLA-A02: 189 | 360.8 |
| HLA-A02: 192 | 162.7 | HLA-A02: 89 | 203.6 | HLA-A02: 138 | 360.8 |
| HLA-A02: 163 | 162.7 | HLA-A02: 93 | 203.6 | HLA-A02: 228 | 360.8 |
| HLA-A02: 221 | 162.7 | HLA-A02: 95 | 203.6 | HLA-A02: 260 | 360.8 |
| HLA-A02: 159 | 162.7 | HLA-A02: 96 | 203.6 | HLA-A02: 107 | 360.8 |
| HLA-A02: 194 | 162.7 | HLA-A02: 97 | 203.6 | HLA-A02: 215 | 360.8 |
| HLA-A02: 140 | 162.7 | HLA-A02: 107 | 203.6 | HLA-A02: 182 | 360.8 |
| HLA-A02: 206 | 162.7 | HLA-A02: 109 | 203.6 | HLA-A02: 09 | 360.8 |
| HLA-A02: 74 | 162.7 | HLA-A02: 111 | 203.6 | HLA-A02: 192 | 360.8 |
| HLA-A02: 198 | 162.7 | HLA-A02: 118 | 203.6 | HLA-A02: 163 | 360.8 |
| HLA-A02: 123 | 162.7 | HLA-A02: 119 | 203.6 | HLA-A02: 221 | 360.8 |
| HLA-A02: 95 | 162.7 | HLA-A02: 120 | 203.6 | HLA-A02: 159 | 360.8 |
| HLA-A02: 168 | 162.7 | HLA-A02: 173 | 203.6 | HLA-A02: 194 | 360.8 |
| HLA-A02: 150 | 162.7 | HLA-A02: 174 | 203.6 | HLA-A02: 140 | 360.8 |
| HLA-A02: 210 | 162.7 | HLA-A02: 175 | 203.6 | HLA-A02: 206 | 360.8 |
| HLA-A02: 86 | 162.7 | HLA-A02: 176 | 203.6 | HLA-A02: 74 | 360.8 |
| HLA-A02: 235 | 162.7 | HLA-A02: 177 | 203.6 | HLA-A02: 198 | 360.8 |
| HLA-A02: 237 | 162.7 | HLA-A02: 181 | 203.6 | HLA-A02: 123 | 360.8 |
| HLA-A02: 208 | 162.7 | HLA-A02: 212 | 203.6 | HLA-A02: 95 | 360.8 |
| HLA-A02: 212 | 162.7 | HLA-A02: 213 | 203.6 | HLA-A02: 168 | 360.8 |
| HLA-A02: 201 | 162.7 | HLA-A02: 214 | 203.6 | HLA-A02: 150 | 360.8 |
| HLA-A02: 120 | 162.7 | HLA-A02: 215 | 203.6 | HLA-A02: 210 | 360.8 |
| HLA-A02: 240 | 162.7 | HLA-A02: 216 | 203.6 | HLA-A02: 86 | 360.8 |
| HLA-A02: 211 | 162.7 | HLA-A02: 218 | 203.6 | HLA-A02: 235 | 360.8 |
| HLA-A02: 175 | 162.7 | HLA-A02: 220 | 203.6 | HLA-A02: 237 | 360.8 |
| HLA-A02: 162 | 162.7 | HLA-A02: 221 | 203.6 | HLA-A02: 208 | 360.8 |
| HLA-A02: 121 | 162.7 | HLA-A02: 202 | 203.6 | HLA-A02: 212 | 360.8 |
| HLA-A02: 89 | 162.7 | HLA-A02: 203 | 203.6 | HLA-A02: 201 | 360.8 |
| HLA-A02: 220 | 162.7 | HLA-A02: 204 | 203.6 | HLA-A02: 120 | 360.8 |
| HLA-A02: 164 | 162.7 | HLA-A02: 205 | 203.6 | HLA-A02: 240 | 360.8 |
| HLA-A02: 190 | 162.7 | HLA-A02: 206 | 203.6 | HLA-A02: 211 | 360.8 |
| HLA-A02: 157 | 162.7 | HLA-A02: 207 | 203.6 | HLA-A02: 175 | 360.8 |
| HLA-A02: 96 | 162.7 | HLA-A02: 208 | 203.6 | HLA-A02: 162 | 360.8 |
| HLA-A02: 256 | 162.7 | HLA-A02: 210 | 203.6 | HLA-A02: 121 | 360.8 |
| HLA-A02: 234 | 162.7 | HLA-A02: 211 | 203.6 | HLA-A02: 89 | 360.8 |
| HLA-A02: 97 | 162.7 | HLA-A02: 237 | 203.6 | HLA-A02: 220 | 360.8 |
| HLA-A02: 204 | 162.7 | HLA-A02: 238 | 203.6 | HLA-A02: 164 | 360.8 |
| HLA-A02: 70 | 162.7 | HLA-A02: 239 | 203.6 | HLA-A02: 190 | 360.8 |
| HLA-A02: 77 | 162.7 | HLA-A02: 240 | 203.6 | HLA-A02: 157 | 360.8 |
| HLA-A02: 93 | 162.7 | HLA-A02: 241 | 203.6 | HLA-A02: 96 | 360.8 |
| HLA-A02: 181 | 162.7 | HLA-A02: 132 | 203.6 | HLA-A02: 256 | 360.8 |
| HLA-A02: 111 | 162.7 | HLA-A02: 133 | 203.6 | HLA-A02: 234 | 360.8 |
| HLA-A02: 118 | 162.7 | HLA-A02: 134 | 203.6 | HLA-A02: 97 | 360.8 |
| HLA-A02: 196 | 162.7 | HLA-A02: 138 | 203.6 | HLA-A02: 204 | 360.8 |
| HLA-A02: 185 | 162.7 | HLA-A02: 140 | 203.6 | HLA-A02: 70 | 360.8 |
| HLA-A02: 214 | 162.7 | HLA-A02: 153 | 203.6 | HLA-A02: 77 | 360.8 |
| HLA-A02: 193 | 162.7 | HLA-A02: 157 | 203.6 | HLA-A02: 93 | 360.8 |
| HLA-A02: 200 | 162.7 | HLA-A02: 159 | 203.6 | HLA-A02: 181 | 360.8 |
| HLA-A02: 25 | 162.7 | HLA-A02: 160 | 203.6 | HLA-A02: 111 | 360.8 |
| HLA-A02: 173 | 162.7 | HLA-A02: 162 | 203.6 | HLA-A02: 118 | 360.8 |
| HLA-A02: 177 | 162.7 | HLA-A02: 163 | 203.6 | HLA-A02: 196 | 360.8 |
| HLA-A02: 207 | 162.7 | HLA-A02: 164 | 203.6 | HLA-A02: 185 | 360.8 |
| HLA-A02: 257 | 162.7 | HLA-A02: 165 | 203.6 | HLA-A02: 214 | 360.8 |
| HLA-A02: 203 | 162.7 | HLA-A02: 166 | 203.6 | HLA-A02: 193 | 360.8 |
| HLA-A02: 199 | 162.7 | HLA-A02: 168 | 203.6 | HLA-A02: 200 | 360.8 |
| HLA-A02: 66 | 162.7 | HLA-A02: 251 | 203.6 | HLA-A02: 25 | 360.8 |
| HLA-A02: 01 | 162.7 | HLA-A02: 252 | 203.6 | HLA-A02: 173 | 360.8 |
| HLA-A02: 216 | 162.7 | HLA-A02: 256 | 203.6 | HLA-A02: 177 | 360.8 |
| HLA-A02: 133 | 162.7 | HLA-A02: 257 | 203.6 | HLA-A02: 207 | 360.8 |
| HLA-A02: 119 | 162.7 | HLA-A02: 145 | 203.6 | HLA-A02: 257 | 360.8 |
| HLA-A02: 153 | 162.7 | HLA-A02: 149 | 203.6 | HLA-A02: 203 | 360.8 |
| HLA-A02: 251 | 162.7 | HLA-A02: 150 | 203.6 | HLA-A02: 199 | 360.8 |
| HLA-A02: 145 | 162.7 | HLA-A02: 192 | 203.6 | HLA-A02: 66 | 360.8 |
| HLA-A02: 24 | 162.7 | HLA-A02: 193 | 203.6 | HLA-A02: 01 | 360.8 |
| HLA-A02: 197 | 162.7 | HLA-A02: 194 | 203.6 | HLA-A02: 216 | 360.8 |
| HLA-A02: 236 | 162.7 | HLA-A02: 196 | 203.6 | HLA-A02: 133 | 360.8 |
| HLA-A02: 149 | 162.7 | HLA-A02: 197 | 203.6 | HLA-A02: 119 | 360.8 |
| HLA-A02: 68 | 162.7 | HLA-A02: 198 | 203.6 | HLA-A02: 153 | 360.8 |
| HLA-A02: 218 | 162.7 | HLA-A02: 199 | 203.6 | HLA-A02: 251 | 360.8 |
| HLA-A02: 205 | 162.7 | HLA-A02: 200 | 203.6 | HLA-A02: 145 | 360.8 |
| HLA-A02: 31 | 162.7 | HLA-A02: 201 | 203.6 | HLA-A02: 24 | 360.8 |
| HLA-A02: 239 | 162.7 | HLA-A02: 228 | 203.6 | HLA-A02: 197 | 360.8 |
| HLA-A02: 109 | 162.7 | HLA-A02: 234 | 203.6 | HLA-A02: 236 | 360.8 |
| HLA-A02: 67 | 162.7 | HLA-A02: 235 | 203.6 | HLA-A02: 149 | 360.8 |
| HLA-A02: 132 | 162.7 | HLA-A02: 236 | 203.6 | HLA-A02: 68 | 360.8 |
| HLA-A02: 134 | 162.7 | HLA-A02: 260 | 203.6 | HLA-A02: 218 | 360.8 |
| HLA-A02: 252 | 162.7 | HLA-A02: 266 | 203.6 | HLA-A02: 205 | 360.8 |
| HLA-A02: 202 | 162.7 | HLA-A02: 182 | 203.6 | HLA-A02: 31 | 360.8 |
| HLA-A02: 213 | 162.7 | HLA-A02: 183 | 203.6 | HLA-A02: 239 | 360.8 |
| HLA-A02: 35 | 163.8 | HLA-A02: 185 | 203.6 | HLA-A02: 109 | 360.8 |
| HLA-A02: 161 | 166.2 | HLA-A02: 187 | 203.6 | HLA-A02: 67 | 360.8 |
| HLA-A02: 245 | 166.6 | HLA-A02: 189 | 203.6 | HLA-A02: 132 | 360.8 |
| HLA-A02: 73 | 166.6 | HLA-A02: 190 | 203.6 | HLA-A02: 134 | 360.8 |
| HLA-A02: 105 | 172.3 | HLA-A02: 121 | 203.6 | HLA-A02: 252 | 360.8 |
| HLA-A02: 12 | 172.7 | HLA-A02: 123 | 203.6 | HLA-A02: 202 | 360.8 |
| HLA-A02: 27 | 189.1 | HLA-A02: 161 | 208.6 | HLA-A02: 213 | 360.8 |
| HLA-A02: 148 | 198.3 | HLA-A02: 35 | 211 | HLA-A02: 161 | 371 |
| HLA-A02: 139 | 200.4 | HLA-A02: 38 | 216.6 | HLA-A02: 122 | 376.5 |
| HLA-A02: 78 | 212.1 | HLA-A02: 139 | 240 | HLA-A02: 27 | 392.6 |
| HLA-A02: 262 | 213.2 | HLA-A02: 262 | 240.9 | HLA-A02: 262 | 405 |
| HLA-A02: 38 | 221.4 | HLA-A02: 41 | 247.7 | HLA-A02: 233 | 412.4 |
| HLA-A02: 41 | 221.5 | HLA-A02: 58 | 279.9 | HLA-A02: 41 | 425.7 |
| HLA-A02: 167 | 230.1 | HLA-A02: 233 | 288.9 | HLA-A02: 139 | 439.8 |
| HLA-A02: 58 | 235.2 | HLA-A02: 147 | 299.3 | HLA-A02: 44 | 468.5 |
| HLA-A02: 34 | 239.2 | HLA-A02: 151 | 299.3 | HLA-A02: 142 | 468.5 |
| HLA-A02: 20 | 251.9 | HLA-A02: 167 | 305.1 | HLA-A02: 58 | 470.4 |
| HLA-A02: 233 | 261.8 | HLA-A02: 20 | 309.4 | HLA-A02: 229 | 474.1 |
| HLA-A02: 147 | 275.3 | HLA-A02: 122 | 312.8 | HLA-A02: 167 | 486 |
| HLA-A02: 151 | 275.3 | HLA-A02: 44 | 325.5 | HLA-A02: 147 | 495.7 |
| HLA-A02: 42 | 289.3 | HLA-A02: 142 | 325.5 | HLA-A02: 151 | 495.7 |
| HLA-A02: 60 | 324.7 | HLA-A02: 34 | 332.2 | | |
| HLA-A02: 62 | 337.7 | HLA-A02: 42 | 340.2 | | |
| HLA-A02: 126 | 345.7 | HLA-A02: 78 | 363.6 | | |
| HLA-A02: 51 | 345.7 | HLA-A02: 06 | 369.7 | | |
| HLA-A02: 61 | 345.7 | HLA-A02: 21 | 369.7 | | |
| HLA-A02: 79 | 345.7 | HLA-A02: 28 | 369.7 | | |
| HLA-A02: 137 | 345.7 | HLA-A02: 51 | 369.7 | | |
| HLA-A02: 170 | 345.7 | HLA-A02: 61 | 369.7 | | |
| HLA-A02: 06 | 345.7 | HLA-A02: 72 | 369.7 | | |
| HLA-A02: 28 | 345.7 | HLA-A02: 79 | 369.7 | | |
| HLA-A02: 72 | 345.7 | HLA-A02: 91 | 369.7 | | |
| HLA-A02: 259 | 345.7 | HLA-A02: 106 | 369.7 | | |
| HLA-A02: 180 | 345.7 | HLA-A02: 180 | 369.7 | | |
| HLA-A02: 91 | 345.7 | HLA-A02: 137 | 369.7 | | |
| HLA-A02: 248 | 345.7 | HLA-A02: 170 | 369.7 | | |
| HLA-A02: 106 | 345.7 | HLA-A02: 248 | 369.7 | | |
| HLA-A02: 144 | 345.7 | HLA-A02: 144 | 369.7 | | |
| HLA-A02: 21 | 345.7 | HLA-A02: 259 | 369.7 | | |
| HLA-A02: 44 | 358.3 | HLA-A02: 126 | 369.7 | | |
| HLA-A02: 142 | 358.3 | HLA-A02: 243 | 379.8 | | |
| HLA-A02: 122 | 371.1 | HLA-A02: 52 | 398.7 | | |
| HLA-A02: 48 | 372 | HLA-A02: 48 | 418.4 | | |
| HLA-A02: 127 | 388.2 | HLA-A02: 60 | 421.2 | | |
| HLA-A02: 52 | 391.1 | HLA-A02: 62 | 473.9 | | |
| HLA-A02: 254 | 434.1 | HLA-A02: 127 | 479.9 | | |
| HLA-A02: 243 | 457.3 | HLA-A02: 229 | 487.6 | | |
| HLA-A02: 224 | 458.7 | | | | |
| HLA-A02: 36 | 469 | | | | |
| HLA-A02: 169 | 471.5 | | | | |
| HLA-A02: 101 | 486.1 | | | | |

### Example 4—Recombinant T-Cell Receptors

Candidate T-cell clones were then subjected to alpha-beta TCR amplification and sequencing. It was determined that KTCR-1 had the TRAV27*01 allele (SEQ ID NO:5 DNA and SEQ ID NO:6 amino acid) as the sequence for the variable region of the alpha chain of the TCR and the TRBV19*01 allele (SEQ ID NO:7 DNA and SEQ ID NO:8 amino acid) as the sequence for the beta chain of the TCR; that KTCR-2 had the TRAV13-2*01 allele (SEQ ID NO:9 DNA and SEQ ID NO:10 amino acid) as the sequence for the variable region of the alpha chain of the TCR and the TRBV19*01 allele (SEQ ID NO:7 DNA and SEQ ID NO:8 amino acid) as the sequence for the variable region of the beta chain of the TCR, and that KTCR-3 had the TRAV27*01 allele (SEQ ID NO:5 DNA and SEQ ID NO:6 amino acid) as the sequence for the variable region of the alpha chain of the TCR and the TRBV4-1*01 alelle (SEQ ID NO:11 DNA and SEQ ID NO:12 amino acid) as the sequence for the variable region of the beta chain of the TCR.

The alleles identified in the alpha and beta chains of the TCRs identified from KTCR-1, KTCR-2 and KTCR-3 are shown below in Table 3, along with the binding specificity of each (i.e. KRAS^{G12D} or KRAS^{G12V}). Based on these results, it is predicted that a TCR having the variable chain regions of TRAV13-2*01 for the alpha chain and TRBV04-1*01 for the beta chain of the TCR should also be effective in binding to KRASGI2X mutant peptides as presented by HLA-A*02:01. Such a construct is referred to herein as PTCR-4 as a predicted construct. Without being bound by theory, it is predicted that the PTCR-4 construct would recognize HLA-A*02:01 restricted KRAS^{G12D} and KRAS^{G12V}, but not KRAS^{Wild Type}.

TABLE 3
Alleles for variable chain region of alpha and
beta chains of sequenced TCRs.

| Alpha Chain | Beta Chain Variable Region | |
|---|---|---|
| Variable Region | TRBV 19*01 | TRBV 04-1*01 |
| TRAV27*01 | KTCR-1 | KTCR-3 |
| | (KRAS^{G12V}) | (KRAS^{G12D}) |
| TRAV13-2*01 | KTCR-2 | Predicted |
| | (KRAS^{G12D}) | (PTCR-4) |

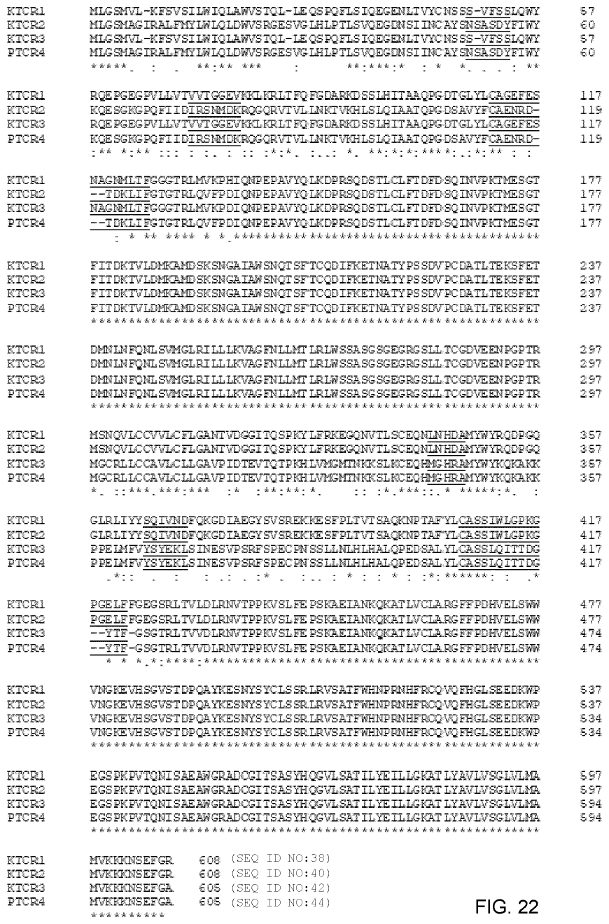

The variable region of each of the alpha and beta chains of the TCR containing the foregoing alleles contains the first and second complementarity determining region (CDR) of each chain (CDR1 and CDR2). The sequence of the third CDR was determined for each of KTCR-1, KTCR-2 and KTCR-3 to identify the sequences of each of the complementarity determining regions as follows in Table 4 and as underlined in FIG. 22.

TABLE 4
Amino acid sequences of the first, second and third CDRs for each alpha and beta chain of
each TCR.

| | KTCR-1 | KTCR-2 | KTCR-3 | |
|---|---|---|---|---|
| | (KRAS^{G12V}) | (KRAS^{G12D}) | (KRAS^{G12D}) | PTCR-4 |
| CDR1-alpha | SEQ ID NO: 14 | SEQ ID NO: 18 | SEQ ID NO: 14 | SEQ ID NO: 18 |
| CDR2-alpha | SEQ ID NO: 16 | SEQ ID NO: 20 | SEQ ID NO: 16 | SEQ ID NO: 20 |
| CDR3-alpha | SEQ ID NO: 30 | SEQ ID NO: 34 | SEQ ID NO: 30 | SEQ ID NO: 34 |
| CDR1-beta | SEQ ID NO: 22 | SEQ ID NO: 22 | SEQ ID NO: 26 | SEQ ID NO: 26 |
| CDR2-beta | SEQ ID NO: 24 | SEQ ID NO: 24 | SEQ ID NO: 28 | SEQ ID NO: 28 |
| CDR3-beta | SEQ ID NO: 32 | SEQ ID NO: 32 | SEQ ID NO: 36 | SEQ ID NO: 36 |

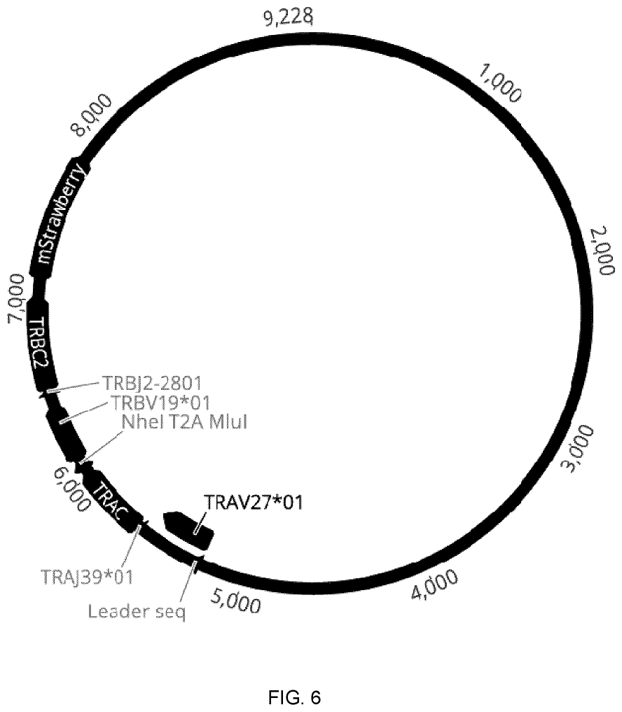

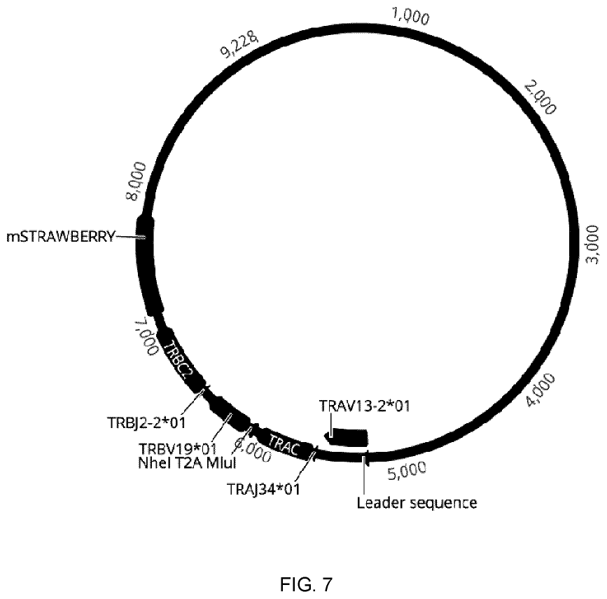

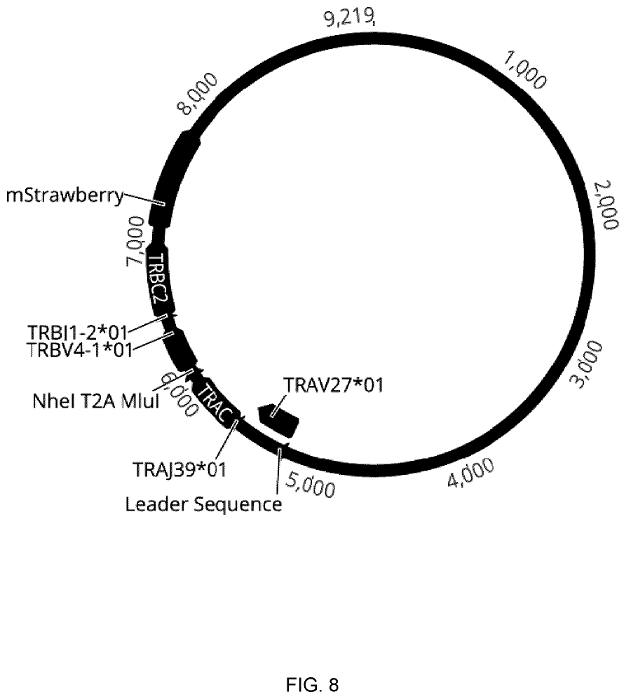

Recombinant TCRs for reconstitution were designed, incorporating the novel alpha-beta TCR sequences from the above three distinct T-cell clones, KTCR-1, KTCR-2 and KTCR-3, respectively. Physical DNA was synthesized de novo according to these designs, then ligated into lentiviral transfer plasmids shown schematically in FIGS. 6-8 (corresponding to SEQ ID NOs:45, 46 and 47, with the predicted plasmid sequence to generate PTCR-4 shown as SEQ ID NO:48).

### Example 5—Engineered CD8+ T Cells

Replication-incompetent lentiviral particles were then generated as TCR gene transfer vectors and used to transduce healthy donor CD8⁺ T-cells.

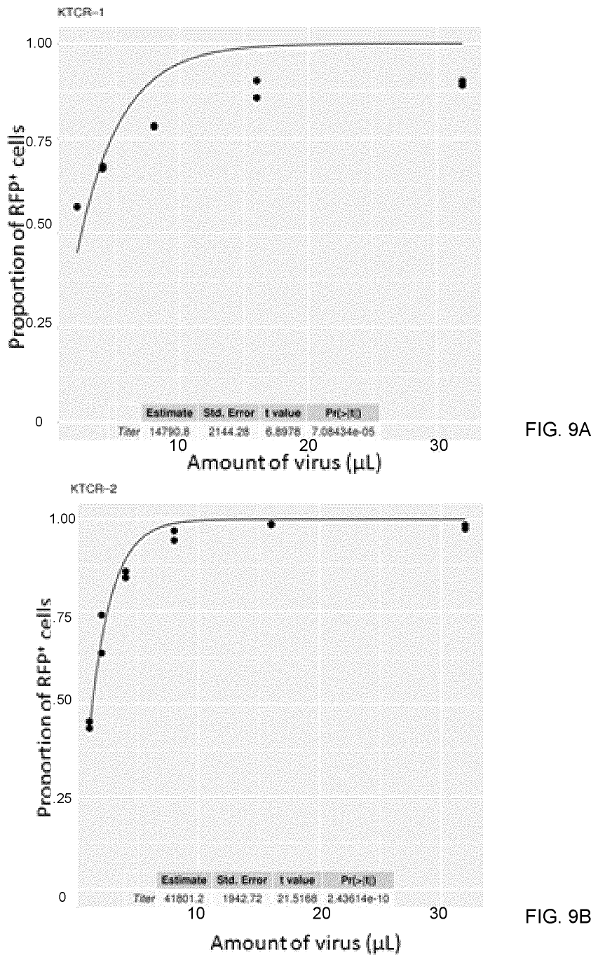

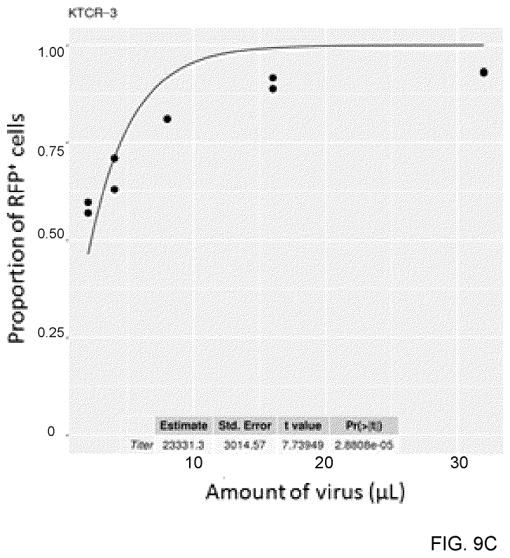

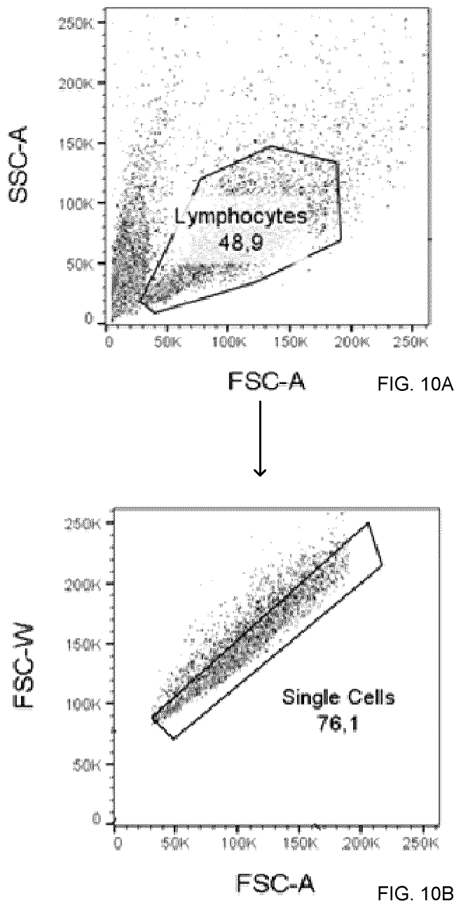

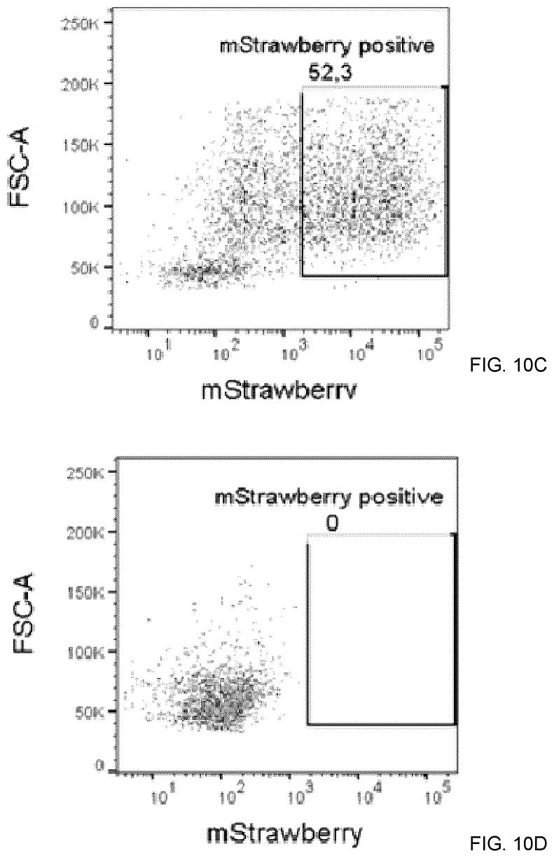

FIGS. 9A, 9B and 9C show the results of KTCR-1, KTCR-2, and KTCR-3 lentivirus titration over HeLa cells. Varying amounts of each lentivirus were added to 5×10⁴ HeLa cells for 48 hours. The HeLa cells were then analysed for red fluorescent protein (reporter gene, mStrawberry) expression using flow cytometry (example shown in FIGS. 10A, 10B, 10C and 10D, mStrawberry positive cells shown in FIG. 10C), to determine an optimal amount of the lentivirus required in future transfections.

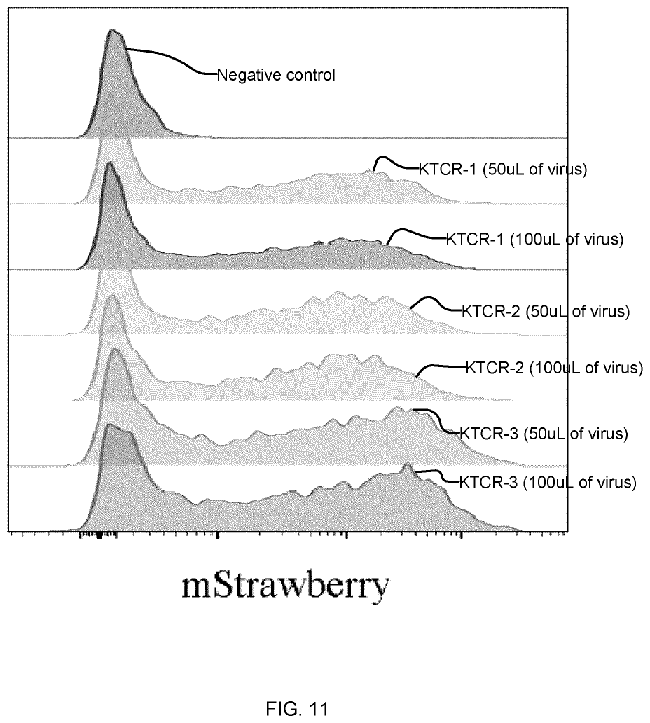

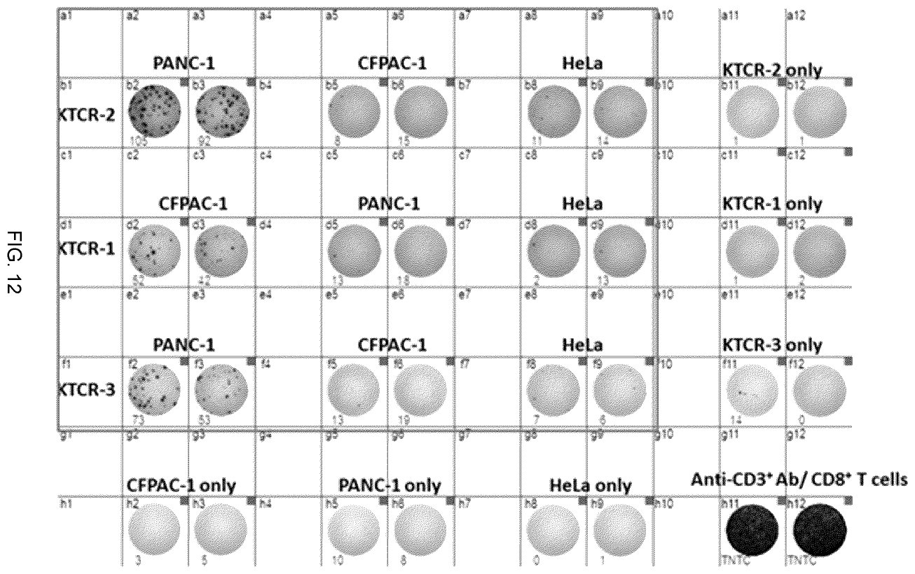

FIG. 11 shows the results of sorting KTCR-1, KTCR-2 and KTCR-3 transduced CD8⁺ T cells. A flow gating procedure was followed to isolate CD8⁺ T cells expressing the reporter gene, mStrawberry, post KTCR-1, KTCR-2, and KTCR-3 lentiviral transfection after initial expansion. Shown is a labelled histogram showing the mStrawberry positives compared to the negative control. CD8⁺ T cells were isolated using magnetic bead based cell isolation kit, following the manufacturer's protocol (Miltenyi Biotec, Bergisch Gladbach, Germany). CD8⁺ T-cells were then activated using anti-CD3 and anti-CD28 antibodies (BioLegend San Diego, Calif., USA) at a final concentration of 1 μg/mL. 24 hours post activation, CD8⁺ T-cells were counted and plated into a 12-well culture plate (Thermo Fisher, CA. USA) at a predetermined concentration of cells in order to achieve a multiplicity of infection (MOI) of 1 and 2 by adding either 50 and 100 μL of each virus to the relevant cells, respectively. 48 hours after transfection, cells were resuspended in supplemented RPMI-1640 media (Thermo Fisher, CA. USA) with 300U/mL of rIL-2 (PreproTech, NJ. USA) and irradiated (50 Gy) feeder PBMCs, at a ratio of 1:100 (transfected CD8⁺ T cells:irradiated feeder cells). After 1 week of expansion, cells were sorted as per the flow gating protocol.

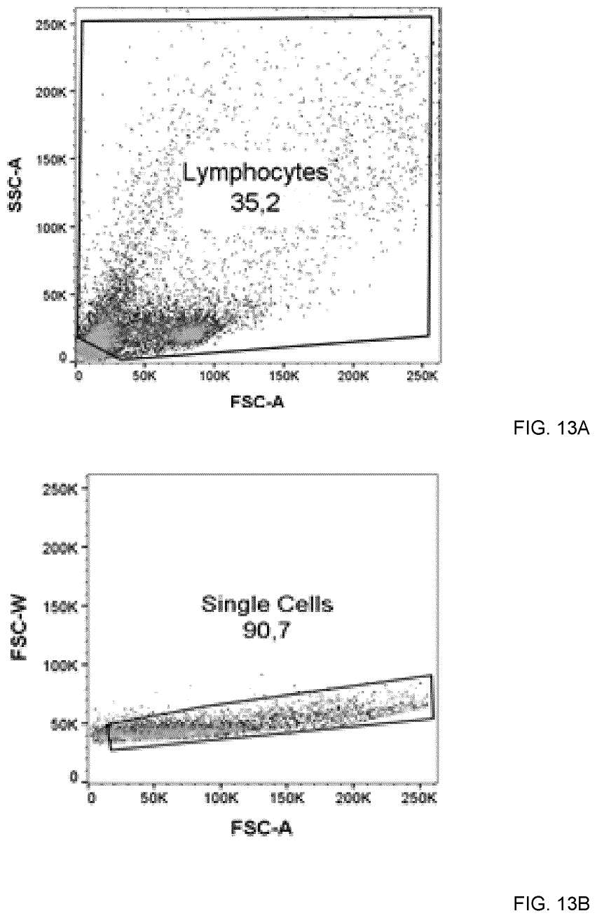

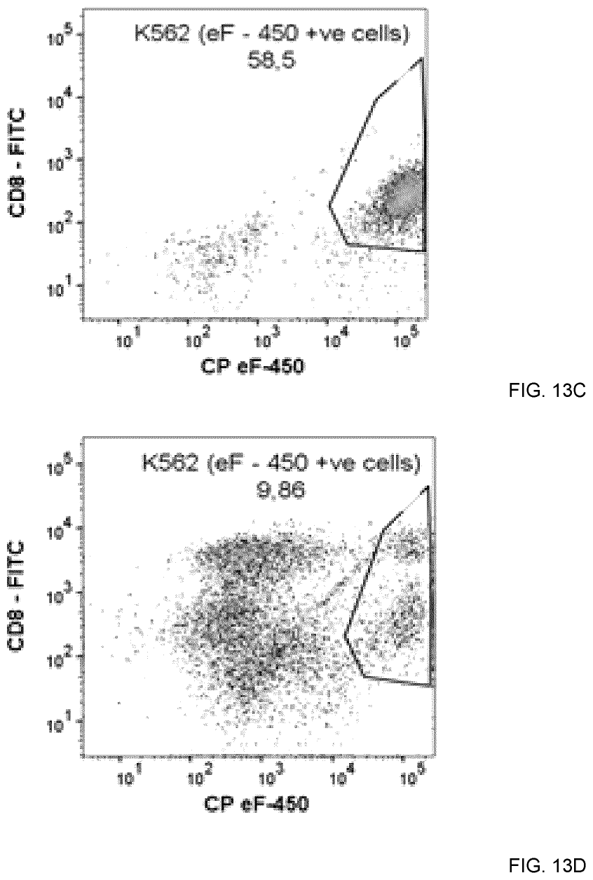

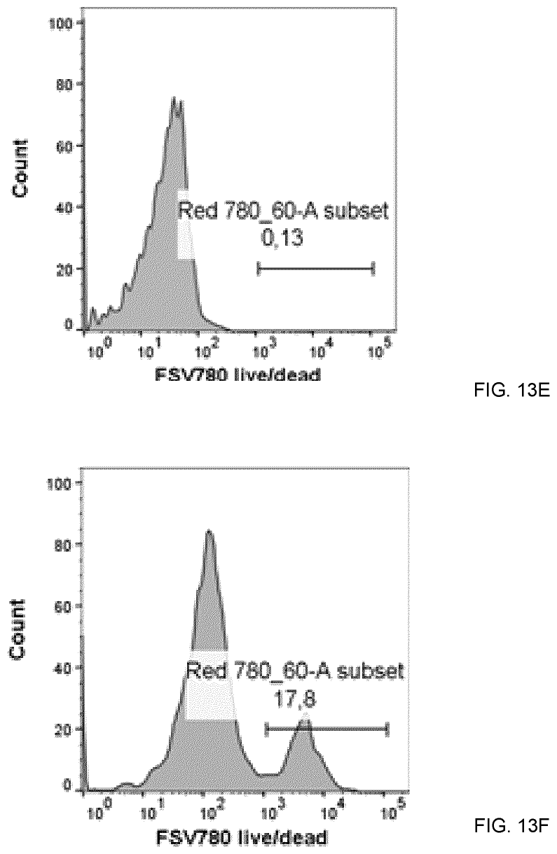

TCR-transduced CD8⁺ T cells were then evaluated for anti-KRAS^{G12X} function and specificity by ELISPOT (as shown in FIG. 12 and Table 5) and cytotoxicity against HLA-A*02:01/KRAS^{G12X} positive target cells (as shown in FIGS. 13A-13F, 14 and 15 and Table 6). By the procedures described above three distinct, validated anti-KRAS^{G12X} TCRs were obtained (KTCR-1, KTCR-2 and KTCR-3).

FIG. 12 shows raw ELISpot data that was analysed using Graphpad—Prism 8 (version 8.0.0). As shown, KTCR-1 CD8⁺ T cells showed an increase in gamma interferon (IFNγ) spot forming units (SFU) when co-cultured with HLA-A*02:01⁺ KRAS^{G12V} CFPAC-1 cells, when compared to the HLA-A*02:01⁺ KRAS^{G12D} PANC-1 and HLA-A*02:01⁻ KRAS^{Wild type} HeLa cells. Similarly, the KTCR-2, and KTCR-3 CD8⁺ T cells showed an increase in IFNγ SFUs when co-cultured with HLA-A*02:01⁺ KRAS^{G12D} PANC-1 when compared to HLA-A*02:01⁺ KRAS^{G12V} CFPAC-1 and HLA-A*02:01⁻ KRAS^{Wild type} HeLa cells.

Table 5 shows the results from ELISpot analysis of KTCR-1, KTCR-2, and KTCR-3 CD8⁺ T-cells. The results were reported as spot forming units (SFU) of gamma interferon (IFNγ). An ANOVA statistical analysis and a follow-up multiple comparison (Tukey's HSD multiple comparison test) were performed. A significant variance was found between KTCR-1 CD8⁺ T cells when co-cultured with HLA-A*02:01⁺ KRAS^{G12V} CFPAC-1 cells, compared to the HLA-A*02:01⁻ KRAS^{Wild type} HeLa cells. Similarly, the KTCR-2, and KTCR-3 CD8⁺ T cells showed a significant increase in IFNγ SFUs when co-cultured with HLA-A*02:01⁺ KRAS^{G12D} PANC-1 when compared to HLA-A*02:01⁺ KRAS^{G12V} CFPAC-1 and HLA-A*02:01⁻ KRAS^{Wild type} HeLa cells. Data analysis was performed using Graphpad—Prism 8 (version 8.0.0).

TABLE 5
Analysis of KTCR-1, KTCR-2 and KTCR-3 CD8⁺ T-cells.

| | | | | | |
|---|---|---|---|---|---|
| SFU of IFNγ/ | | | | | |
| 2.0 × 10⁴ cell | | | | | Multiple comparison |
| input | Mean | SD | N | ANOVA | test |

KTCR-1

| | | | | | | | | |
|---|---|---|---|---|---|---|---|---|
| PANC-1 | 13 | 18 | 19.5 | 9.19 | 2 | P = 0.016 | vs | |
| HLA-A*02:01⁺ KRAS^{G12D} | | | | | | | HeLa | |
| | | | | | | | p = 0.818 | |
| CFPAC-1 | 52 | 42 | 42.0 | 14.14 | 2 | | vs | Vs |
| HLA-A*02:01⁺ KRAS^{G12V} | | | | | | | PANC-1 | HeLa |
| | | | | | | | p = 0.025 | p = 0.019 |
| HeLa | 2 | 13 | 7.5 | 7.78 | 2 | | | |
| HLA-A*02:01⁺ KRAS^{wild type} | | | | | | | | |

KTCR-2

| | | | | | | | | |
|---|---|---|---|---|---|---|---|---|
| PANC-1 | 105 | 92 | 98.5 | 9.19 | 2 | P < 0.001 | vs | vs |
| HLA-A*02:01⁺ KRAS^{G12D} | | | | | | | CFPAC-1 | HeLa |
| | | | | | | | p = 0.002 | p = 0.002 |
| CFPAC-1 | 8 | 15 | 11.5 | 4.95 | 2 | | vs | |
| HLA-A*02:01⁺ KRAS^{G12V} | | | | | | | HeLa | |
| | | | | | | | p = 0.882 | |
| HeLa | 11 | 14 | 12.5 | 2.12 | 2 | | | |
| HLA-A*02:01⁺ KRAS^{wild type} | | | | | | | | |

KTCR-3

| | | | | | | | | |
|---|---|---|---|---|---|---|---|---|
| PANC-1 | 73 | 53 | 63.0 | 14.14 | 2 | P = 0.016 | vs | vs |
| HLA-A*02:01⁺ KRAS^{G12D} | | | | | | | CFPAC-1 | HeLa |
| | | | | | | | p = 0.029 | p = 0.018 |
| CFPAC-1 | 13 | 19 | 21.0 | 2.83 | 2 | | vs | |
| HLA-A*02:01⁺ KRAS^{G12V} | | | | | | | HeLa | |
| | | | | | | | p = 0.628 | |
| HeLa | 7 | 6 | 6.5 | 0.71 | 2 | | | |
| HLA-A*02:01⁺ KRAS^{wild type} | | | | | | | | |

FIGS. 13A-13D show exemplary flow cytometry data analysis of K562-A*02:01 cells pulsed with KRAS^{G12D} peptide and co-cultured with KTCR-2 cells and control lymphocytes. A flow cytometry gating protocol was followed. ef450 stained (eBiosciences, Thermo Fisher, CA. USA) proliferated K562-A*02:01 cells were gated to include those double positive for FITC-CD8 (eBiosciences, Thero Fisher, CA. USA). This selection assumed the double positive staining was due to effector CD8⁺ T-cells being bound to the target ef450 stained K562-A*02:01 cells at the time of analysis and not that the K562-A*02:01 cells were also expressing CD8⁺ T cells. This was confirmed when comparing the K562-A*02:01 pulsed with KRAS^{G12D} peptide and co-cultured KTCR-2 cells (FIG. 13F) and control lymphocytes (FIG. 13E) to evaluate cytotoxic activity of the KTCR-2 cells against the pulsed cells. Cells were cultured in RPMI-1640 supplemented media (Thermo Fisher, CA. USA).

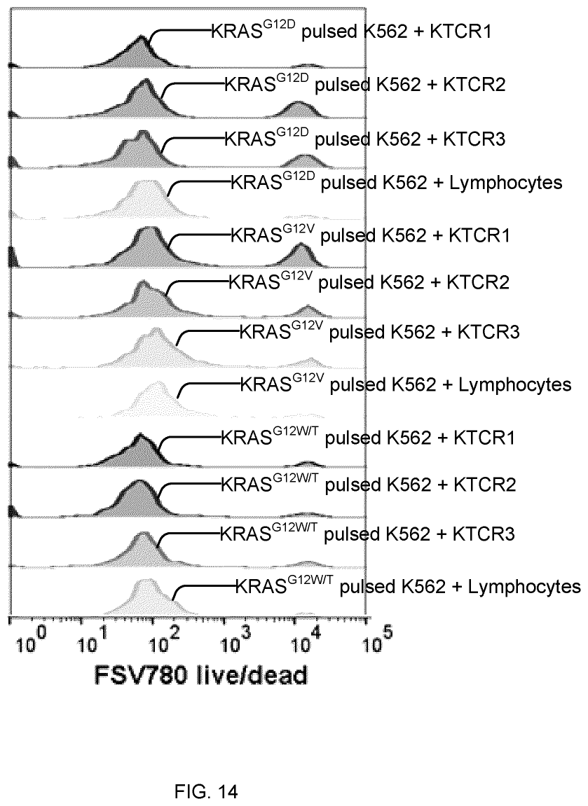

FIG. 14 show the raw data histogram plots of FSV780 (Fixability Viability Stain 780) live/dead stained (BD Biosciences, NJ. USA) K562-A*02:01 cells under the various conditions, using the flow gating procedures outlined with reference to FIGS. 13A-13D.

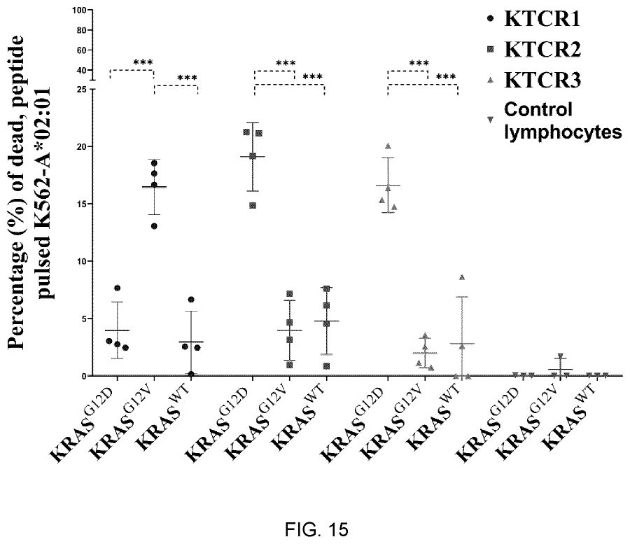

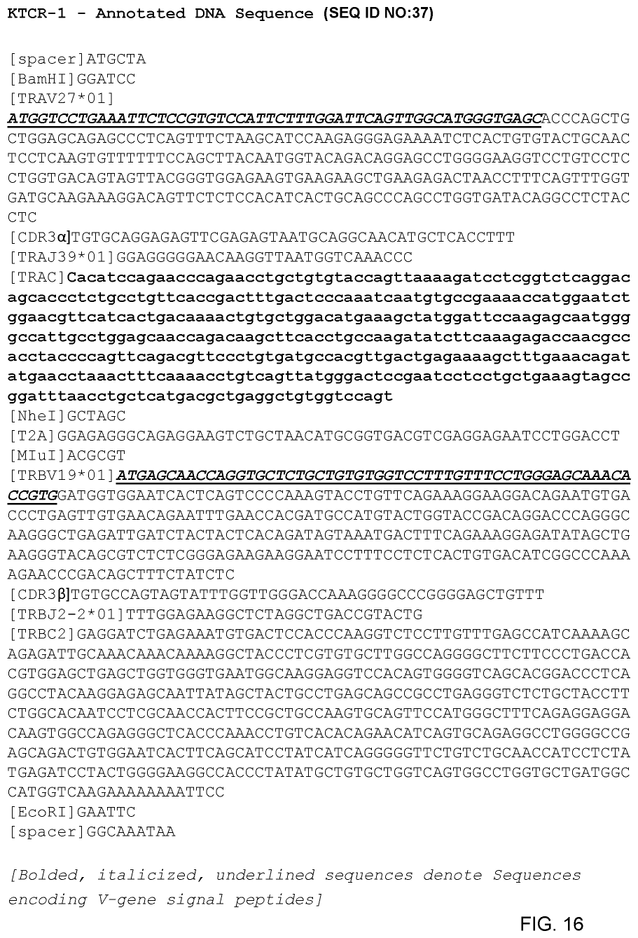

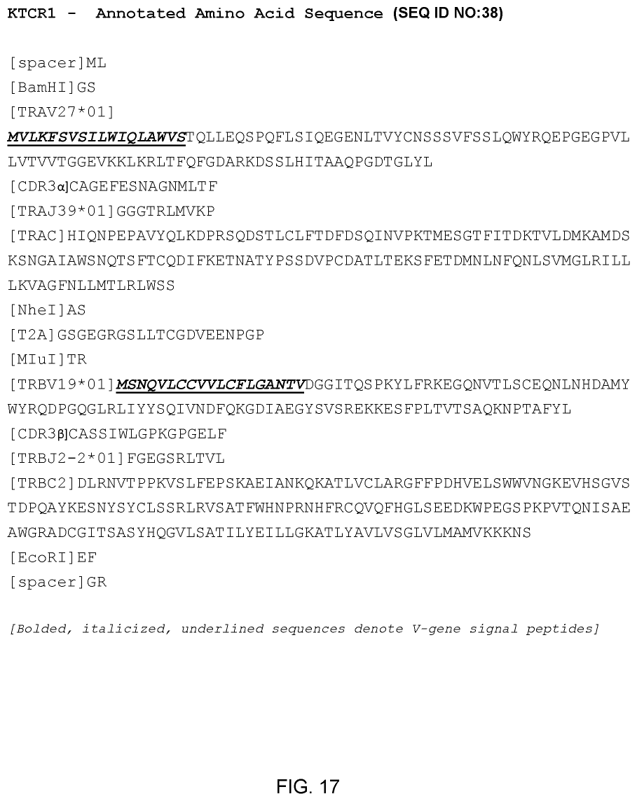

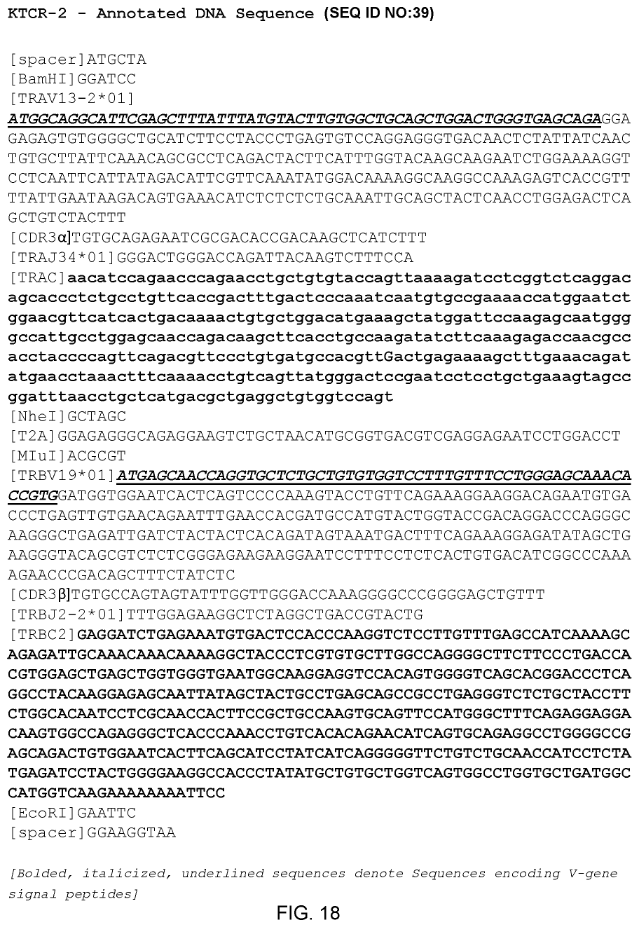

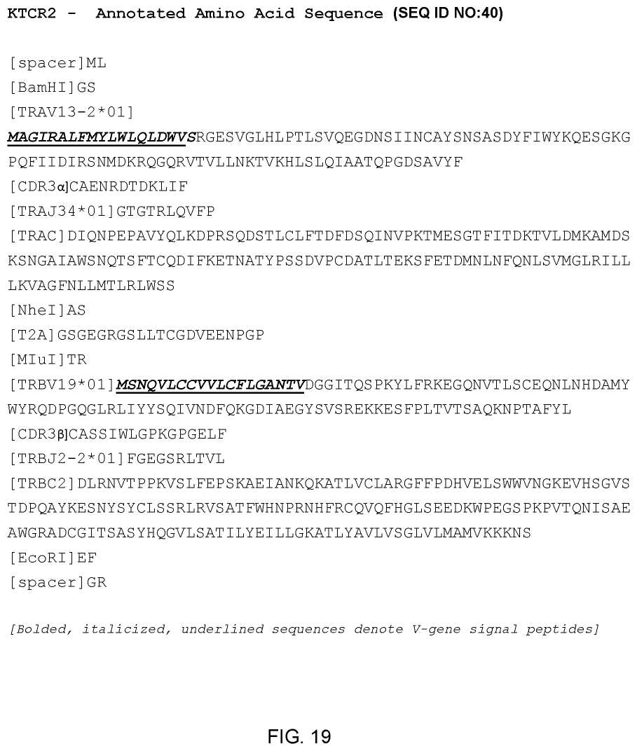

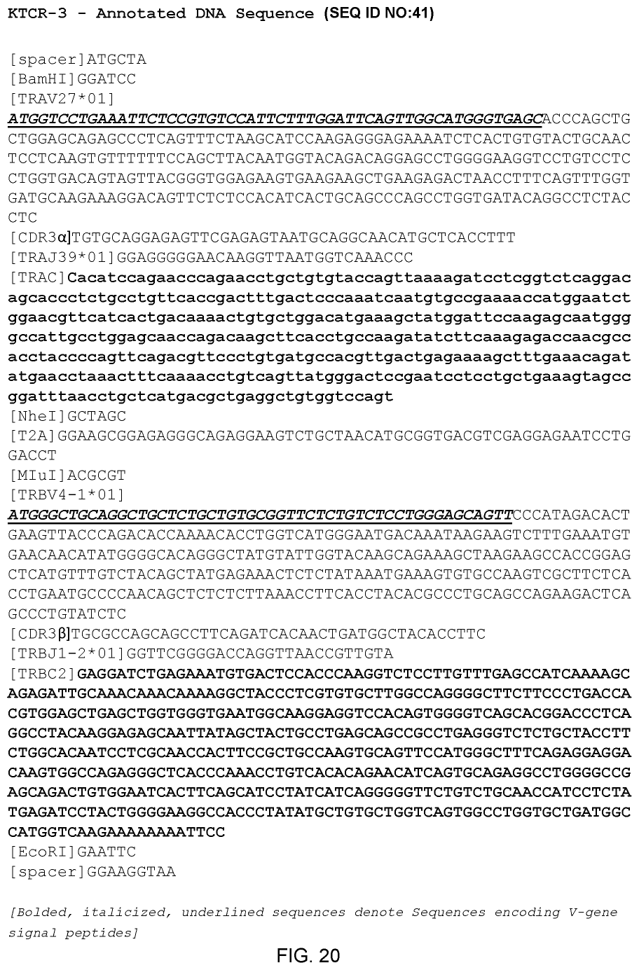

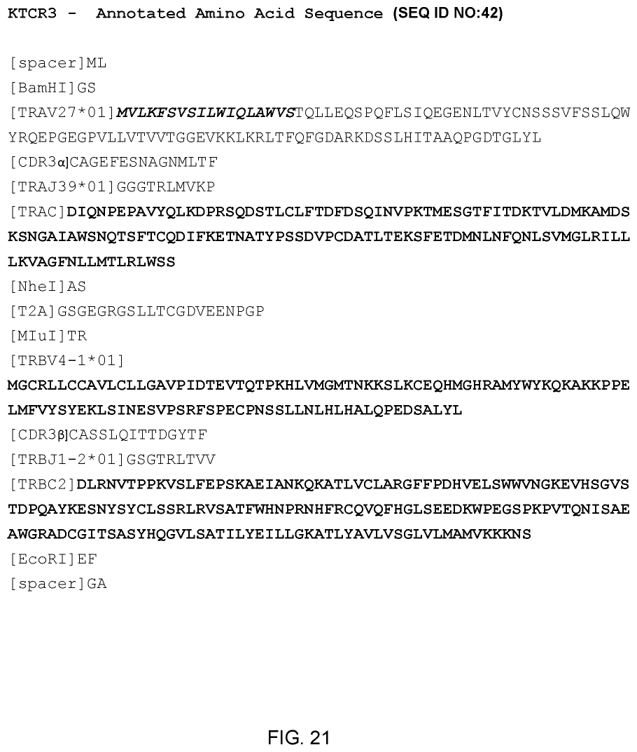

FIG. 15 shows cytolytic assay analysis of the raw data shown in FIG. 14. KTCR1, KRAS^{G12V}-specific, HLA-A*02:01-restricted TCR and KTCR2 and KTCR3, KRAS^{G12D}-specific, HLA-A*02:01-restricted TCRs were co cultured with K562-A*02:01 antigen presenting cells which were peptide pulsed with either the KRAS^{G12D}, KRAS^{G12V}, KRAS^{WT} peptide (10 μg/mL) for 5 hours at an effector to target cell ratio of 5:1. This data was normalised to eliminate non-specific death by comparing the death of the peptide pulsed K562-A*02:01 and unstimulated (not peptide pulsed) K562-A*02:01 when co-cultured with KTCR T cells. KRAS^{G12V} peptide pulsed K562-A*02:01 showed significantly more death as measured by staining with BD Horizon™ Fixable Viability Stain 780, when co-cultured with the KTCR1 T cells (ANOVA, p<0.001, Turkey's multiple comparison test ***P<0.001). The KRAS^{G12D} peptide pulsed K562-A*02:01 showed significantly more death when co-cultured with the KTCR2 or KTCR3 T cells as compared to the KRAS^{G12D} and KRAS^{Wt} pulsed K562-A*02:01 cells (ANOVA, p<0.001 and p=0.272, respectively. Turkey's multiple comparison testing *** p<0.001) Flow analysis was performed using Data analysis was performed using Graphpad—Prism 8 (version 8.0.0).

Table 6 summarizes the data shown in FIG. 15. Statistical analysis using ANOVA shows a significant variance between the mean percentage (%) of cytotoxicity of the target cells, K562-A*02:01 pulsed with the either the KRAS^{G12D}, KRAS^{G12V}, or KRAS_{wild type} epitope and co-cultured with the KTCR-X (i.e. KTCR-1, KTCR-2 or KTCR-3) cells. A multiple comparison (Tukey's HSD multiple comparison test) is also shown and highlights the variance between the mean percentage (%) of cytotoxicity that can be attributed to the specificity of KTCR-2 or KTCR-3 cells to target the HLA-A*02:01 presented KRAS^{G12D} epitope and KTCR-1 cells to target the HLA-A*02:01 presented KRAS^{G12V} epitope. Data analysis was performed using Graphpad—Prism 8 (version 8.0.0).

TABLE 6
Cell lysis of cells pulsed with KRAS^{G12X} peptide and co-cultured with T-cells.

| | | | | |
|---|---|---|---|---|
| Mean % | SD | N | ANOVA | Multiple comparison test |

K562_A*02:01 + KRAS^{G12D}

| | | | | | | | |
|---|---|---|---|---|---|---|---|
| KTCR-1 | 4.0 | 2.47 | 4 | P < 0.001 | vs Control Lymphocytes | | |
| | | | | | p = 0.178 | | |
| KTCR-2 | 19.1 | 2.99 | 4 | | vs KTCR-1 | vs Control Lymphocytes | vs KTCR-3 |
| | | | | | p < 0.001 | p < 0.001 | p = 0.503 |
| KTCR-3 | 16.3 | 2.38 | 4 | | vs KTCR-1 | vs Control Lymphocytes | |
| | | | | | p < 0.001 | p < 0.001 | |
| Control lymphocytes | 0.1 | 0.02 | 4 | | | | |

K562_A*02:01 + KRAS^{G12V}

| | | | | | | | |
|---|---|---|---|---|---|---|---|
| KTCR-1 | 16.5 | 2.41 | 4 | p < 0.001 | vs KTCR-2 | vs KTCR-3 | vs Control |
| | | | | | p < 0.001 | p < 0.001 | Lymphocytes |
| | | | | | | | p < 0.001 |
| KTCR-2 | 4.0 | 2.61 | 4 | | vs Control Lymphocytes | vs KTCR-3 | |
| | | | | | p = 0.292 | p = 0.678 | |
| KTCR-3 | 2.0 | 1.29 | 4 | | vs Control Lymphocytes | | |
| | | | | | p = 0.873 | | |
| Control lymphocytes | 0.6 | 0.97 | 4 | | | | |

K562_A*02:03 + KRAS^{Wild type}

| | | | | | | | |
|---|---|---|---|---|---|---|---|
| KTCR-1 | 2.9 | 2.71 | 4 | p = 0.272 | vs KTCR-2 | vs KTCR-3 | vs Control |
| | | | | | p = 0.723 | p > 0.999 | Lymphocytes |
| | | | | | | | p = 0.419 |
| KTCR-2 | 4.79 | 2.91 | 4 | | vs Control Lymphocytes | vs KTCR-3 | |
| | | | | | p = 0.075 | p = 0.679 | |
| KTCR-3 | 2.82 | 4.08 | 4 | | vs Control Lymphocytes | | |
| | | | | | p = 0.459 | | |
| Control lymphocytes | 0.4 | 0.17 | 4 | | | | |

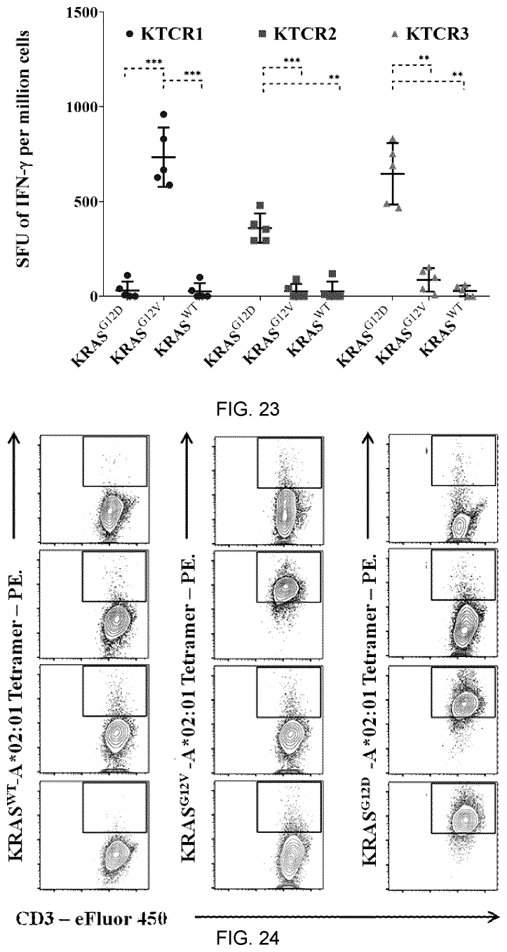

With reference to FIG. 23, K562-A*02:01 cells were pulsed with either the KRAS^{G12D}, KRAS^{G12V}, KRAS^{WT} peptide (10 μg/mL) and then co-cultured with T cells transduced to express the relevant KRAS^{G12X}-specific rTCR and ELISpot performed following manufactures protocols (Mabtech). ANOVA, p=0.0440 and using Tukey's multiple comparison test, the of KRAS^{G12V} specific, HLA-A*02:01-restricted rTCR produced significant IFN-γ spot forming units (SFU) per million cells when co-cultured with the KRAS^{G12V} peptide pulsed K562-A*02:01 cells, compared to KRAS^{G12D} and KRAS^{wt} pulsed K562-A*02:01 cells (*** p=0.0006 and *** p=0.0004, respectively). The KRAS^{G12D} specific, HLA-A*02:01-restricted rTCR showed a significant when co-cultured with the KRAS^{G12D} peptide pulsed K562-A*02:01 cells compared to KRAS^{G12V} and KRAS^{Wt} pulsed K562-A*02:01 cells (**p=0.0015 and **p=0.0023, respectively) K562-A*02:01 cells.

FIG. 24 shows tetramer staining of KRAS^{G12V} and KRAS^{G12D} specific, HLA-A*02:01-restricted TCRs. Bottom three panels shows KRAS^{G12D} specific HLA-A*02:01-restricted TCRs. Middle three panels horizontally show KRAS^{G12V} specific HLA-A*02:01-restricted TCRs. Top three panels show control being T-cells pre-transduction. Tetramers based on the HLA-A*02:01-KRAS^{G12X} peptide complexes were produced by the NIH tetramer core facility (Atlanta, Ga., USA). Over 90% of KRAS^{G12V} specific, HLA-A*02:01-restricted TCR transduced T cells were specifically KRAS^{G12V} Tetramer positive. Over 90% of the KRAS^{G12D} specific, HLA-A*02:01-restricted TCR transduced T cells were specifically KRAS^{G12D} Tetramer positive. The successful transduction and expression of the associated TCR is evident by the positivity shown specifically towards the appropriate tetramer but also in the negative tetramer responses seen in the T cells pre-transduction (top row).

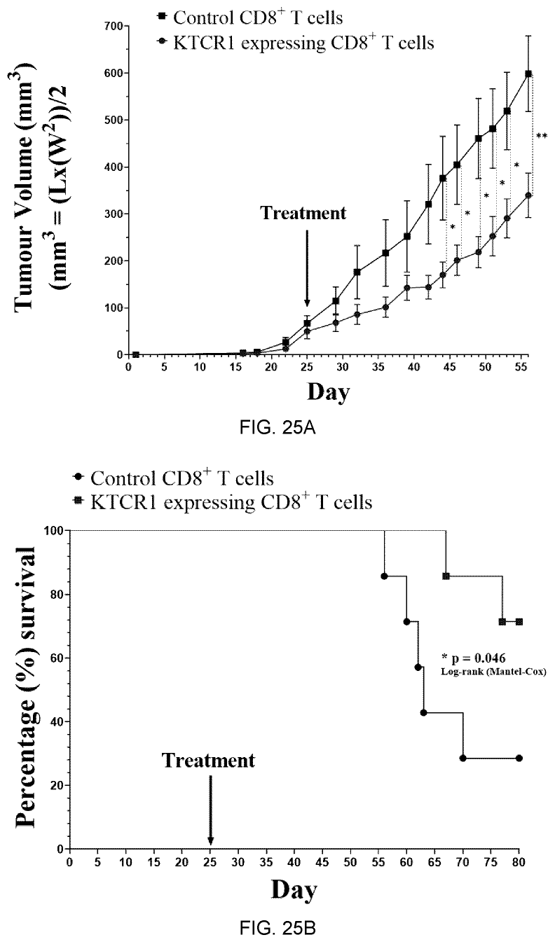

FIG. 25A show the testing results of HLA-A*02:01-restricted KRAS^{G12V} specific TCR reconstituted T cells in vivo. Treatment with the KRAS^{G12V} specific, HLA-A*02:01-restricted T-cells transduced to express KTCR1 significantly reduced growth of KRAS^{G12V}/HLA-A*02:01 patient derived tumors when compared to the mice treated with the control T cells. ANOVA p=0.001 and for multiple comparison, Tukey HSD multiple comparison test, * p<0.018, ** p=0.004. FIG. 25B shows the percentage survival of the treated mice versus the control mice.

The foregoing examples demonstrate that T-cells can be successfully transduced with engineered T-cell receptors that target KRAS^{G12X} mutant peptides restricted and displayed by HLA-A*02:01, and that such T-cells can be used to kill cells that express the KRas having the relevant G12X mutation. Such cells have potential utility in the diagnosis, prophylaxis and/or treatment of cancers in which KRas that is mutated at position 12 is implicated in subjects having the HLA-A*02:01 allele. Based on computational analysis of the predicted binding of KRAS^{G12X} mutant peptides as displayed by other HLA-A*02 alleles, it can be predicted that such cells have potential utility in the diagnosis, prophylaxis and/or treatment of cancers in which KRas that is mutated at position 12 is implicated in subjects having other HLA-A*02 alleles.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

The following references are of interest with respect to the subject matter described herein. The following references and all other references mentioned in this specification are incorporated by reference in their entireties.
1. Jones, S. et al. Core signaling pathways in human pancreatic cancers revealed by global genomic analyses. Science 321, 1801-6 (2008).
2. Weinstein, I. B. Cancer. Addiction to oncogenes—the Achilles heal of cancer.

Science 297, 63-4 (2002).
3. Vonderheide, R. H. & Bayne, L. J. Inflammatory networks and immune surveillance of pancreatic carcinoma. Curr. Opin. Immunol. 25, 200-5 (2013).
4. McAllister, F. et al. Oncogenic Kras activates a hematopoietic-to-epithelial IL-17 signaling axis in preinvasive pancreatic neoplasia. Cancer Cell 25, 621-37 (2014).
5. Winograd, R. et al. Induction of T-cell Immunity Overcomes Complete Resistance to PD-1 and CTLA-4 Blockade and Improves Survival in Pancreatic Carcinoma. Cancer Immunol. Res. 3, 399-411 (2015).
6. Feig, C. et al. Targeting CXCL12 from FAP-expressing carcinoma-associated fibroblasts synergizes with anti-PD-L1 immunotherapy in pancreatic cancer. Proc. Natl. Acad. Sci. U.S.A 110, 20212-7 (2013).
7. Jung, S. & Schluesener, H. J. Human T lymphocytes recognize a peptide of single point-mutated, oncogenic ras proteins. J. Exp. Med. 173, 273-6 (1991).
8. Bergmann-Leitner, E. S., Kantor, J. A., Shupert, W. L., Schlom, J. & Abrams, S. I. Identification of a human CD8+T lymphocyte neo-epitope created by a ras codon 12 mutation which is restricted by the HLA-A2 allele. Cell. Immunol. 187, 103-16 (1998).
9. Kubuschok, B. et al. Naturally occurring T-cell response against mutated p21 ras oncoprotein in pancreatic cancer. Clin. Cancer Res. 12, 1365-72 (2006).
10. Gjertsen, M. K., Bjorheim, J., Saeterdal, I., Myklebust, J. & Gaudernack, G. Cytotoxic CD4+ and CD8+T lymphocytes, generated by mutant p21-ras (12Val) peptide vaccination of a patient, recognize 12Val-dependent nested epitopes present within the vaccine peptide and kill autologous tumour cells carrying this mutation. Int. J. cancer 72, 784-90 (1997).
11. Tran, E. et al. Immunogenicity of somatic mutations in human gastrointestinal cancers. Science 350, 1387-90 (2015).
12. Tran, E. et al. T-Cell Transfer Therapy Targeting Mutant KRAS in Cancer. N. Engl. J. Med. 375, 2255-2262 (2016).
13. Wang, Q. J. et al. Identification of T-cell Receptors Targeting KRAS-Mutated Human Tumors. Cancer Immunol. Res. 4, 204-14 (2016).
14. Sharma, G., Rive, C. M. & Holt, R. A. Rapid selection and identification of functional CD8+ T cell epitopes from large peptide-coding libraries. Nat. Commun. 10, 4553 (2019).
15. Bijen, H. M. et al. Preclinical Strategies to Identify Off-Target Toxicity of High-Affinity TCRs. Mol. Ther. 26, 1206-1214 (2018).
16. Czerkinsky, C. C., Nilsson, L. A., Nygren, H., Ouchterlony, O. & Tarkowski, A. A solid-phase enzyme-linked immunospot (ELISPOT) assay for enumeration of specific antibody-secreting cells. J. Immunol. Methods 65, 109-21 (1983).
17. Janetzki, S. et al. Guidelines for the automated evaluation of Elispot assays. Nat. Protoc. 10, 1098-115 (2015).
18. Dreolini, L. et al. A Rapid and Sensitive Nucleic Acid Amplification Technique for Mycoplasma Screening of Cell Therapy Products. Mol. Ther.—Methods Clin. Dev. (2020). doi:10.1016/j.omtm.2020.01.009
19. Low, J. L., Naidoo, A., Yeo, G., Gehring, A. J., Ho, Z. Z., Yau, Y. H., . . . Grotenbreg, G. M. (2012). Binding of TCR multimers and a TCR-like antibody with distinct fine-specificities is dependent on the surface density of HLA complexes. PloS One, 7(12), e51397. doi:10.1371/journal.pone.0051397.
20. Rydzek, J., Nerreter, T., Peng, H., Jutz, S., Leitner, J., Steinberger, P., . . . Hudecek, M. (2019). Chimeric antigen receptor library screening using a novel NF-κB/NFAT reporter cell platform. Molecular Therapy, 27(2), 287-299. doi:10.1016/j.ymthe.2018.11.015.

1. An antigen targeting agent comprising an antigen binding site that binds to a mutated Kirsten rat sarcoma viral oncogene homolog (KRAS) protein having a missense mutation at position 12 when a peptide incorporating the missense mutation is presented by an HLA-A*02 molecule.

2. An antigen targeting agent as defined in claim 1, wherein the missense mutation at position 12 of the KRAS protein is G12D, G12V or G12C.

3. An antigen targeting agent as defined in claim 1, wherein the HLA-A*02 molecule is HLA-A*02:01.

4. An antigen targeting agent as defined in claim 1, wherein:
the missense mutation at position 12 of the KRAS protein is G12V, and wherein the HLA-A*02 molecule is an HLA-A02:253, HLA-A02:03, HLA-A02:264, HLA-A02:258, HLA-A02:230, HLA-A02:69, HLA-A02:11, HLA-A02:128, HLA-A02:104, HLA-A02:22, HLA-A02:50, HLA-A02:26, HLA-A02:171, HLA-A02:141, HLA-A02:99, HLA-A02:13, HLA-A02:90, HLA-A02:158, HLA-A02:131, HLA-A02:16, HLA-A02:102, HLA-A02:155, HLA-A02:63, HLA-A02:02, HLA-A02:186, HLA-A02:115, HLA-A02:209, HLA-A02:47, HLA-A02:29, HLA-A02:263, HLA-A02:116, HLA-A02:241, HLA-A02:71, HLA-A02:59, HLA-A02:40, HLA-A02:166, HLA-A02:238, HLA-A02:176, HLA-A02:75, HLA-A02:30, HLA-A02:174, HLA-A02:266, HLA-A02:187, HLA-A02:85, HLA-A02:165, HLA-A02:160, HLA-A02:183, HLA-A02:189, HLA-A02:138, HLA-A02:228, HLA-A02:260, HLA-A02:107, HLA-A02:215, HLA-A02:182, HLA-A02:09, HLA-A02:192, HLA-A02:163, HLA-A02:221, HLA-A02:159, HLA-A02:194, HLA-A02:140, HLA-A02:206, HLA-A02:74, HLA-A02:198, HLA-A02:123, HLA-A02:95, HLA-A02:168, HLA-A02:150, HLA-A02:210, HLA-A02:86, HLA-A02:235, HLA-A02:237, HLA-A02:208, HLA-A02:212, HLA-A02:201, HLA-A02:120, HLA-A02:240, HLA-A02:211, HLA-A02:175, HLA-A02:162, HLA-A02:121, HLA-A02:89, HLA-A02:220, HLA-A02:164, HLA-A02:190, HLA-A02:157, HLA-A02:96, HLA-A02:256, HLA-A02:234, HLA-A02:97, HLA-A02:204, HLA-A02:70, HLA-A02:77, HLA-A02:93, HLA-A02:181, HLA-A02:111, HLA-A02:118, HLA-A02:196, HLA-A02:185, HLA-A02:214, HLA-A02:193, HLA-A02:200, HLA-A02:25, HLA-A02:173, HLA-A02:177, HLA-A02:207, HLA-A02:257, HLA-A02:203, HLA-A02:199, HLA-A02:66, HLA-A02:01, HLA-A02:216, HLA-A02:133, HLA-A02:119, HLA-A02:153, HLA-A02:251, HLA-A02:145, HLA-A02:24, HLA-A02:197, HLA-A02:236, HLA-A02:149, HLA-A02:68, HLA-A02:218, HLA-A02:205, HLA-A02:31, HLA-A02:239, HLA-A02:109, HLA-A02:67, HLA-A02:132, HLA-A02:134, HLA-A02:252, HLA-A02:202, HLA-A02:213, HLA-A02:35, HLA-A02:161, HLA-A02:245, HLA-A02:73, HLA-A02:105, HLA-A02:12, HLA-A02:27, HLA-A02:148, HLA-A02:139, HLA-A02:78, HLA-A02:262, HLA-A02:38, HLA-A02:41, HLA-A02:167, HLA-A02:58, HLA-A02:34, HLA-A02:20, HLA-A02:233, HLA-A02:147, HLA-A02:151, HLA-A02:42, HLA-A02:60, HLA-A02:62, HLA-A02:126, HLA-A02:51, HLA-A02:61, HLA-A02:79, HLA-A02:137, HLA-A02:170, HLA-A02:06, HLA-A02:28, HLA-A02:72, HLA-A02:259, HLA-A02:180, HLA-A02:91, HLA-A02:248, HLA-A02:106, HLA-A02:144, HLA-A02:21, HLA-A02:44, HLA-A02:142, HLA-A02:122, HLA-A02:48, HLA-A02:127, HLA-A02:52, HLA-A02:254, HLA-A02:243, HLA-A02:224, HLA-A02:36, HLA-A02:169, or HLA-A02:101 molecule;
the missense mutation at position 12 of the KRAS protein is G12D, and wherein the HLA-A*02 molecule is an HLA-A02:03, HLA-A02:253, HLA-A02:230, HLA-A02:258, HLA-A02:264, HLA-A02:11, HLA-A02:69, HLA-A02:128, HLA-A02:22, HLA-A02:104, HLA-A02:50, HLA-A02:26, HLA-A02:171, HLA-A02:99, HLA-A02:13, HLA-A02:02, HLA-A02:63, HLA-A02:102, HLA-A02:115, HLA-A02:209, HLA-A02:155, HLA-A02:186, HLA-A02:141, HLA-A02:90, HLA-A02:47, HLA-A02:158, HLA-A02:16, HLA-A02:131, HLA-A02:148, HLA-A02:263, HLA-A02:29, HLA-A02:12, HLA-A02:116, HLA-A02:27, HLA-A02:105, HLA-A02:73, HLA-A02:245, HLA-A02:01, HLA-A02:09, HLA-A02:31, HLA-A02:40, HLA-A02:24, HLA-A02:25, HLA-A02:30, HLA-A02:59, HLA-A02:66, HLA-A02:67, HLA-A02:68, HLA-A02:70, HLA-A02:71, HLA-A02:74, HLA-A02:75, HLA-A02:77, HLA-A02:85, HLA-A02:86, HLA-A02:89, HLA-A02:93, HLA-A02:95, HLA-A02:96, HLA-A02:97, HLA-A02:107, HLA-A02:109, HLA-A02:111, HLA-A02:118, HLA-A02:119, HLA-A02:120, HLA-A02:173, HLA-A02:174, HLA-A02:175, HLA-A02:176, HLA-A02:177, HLA-A02:181, HLA-A02:212, HLA-A02:213, HLA-A02:214, HLA-A02:215, HLA-A02:216, HLA-A02:218, HLA-A02:220, HLA-A02:221, HLA-A02:202, HLA-A02:203, HLA-A02:204, HLA-A02:205, HLA-A02:206, HLA-A02:207, HLA-A02:208, HLA-A02:210, HLA-A02:211, HLA-A02:237, HLA-A02:238, HLA-A02:239, HLA-A02:240, HLA-A02:241, HLA-A02:132, HLA-A02:133, HLA-A02:134, HLA-A02:138, HLA-A02:140, HLA-A02:153, HLA-A02:157, HLA-A02:159, HLA-A02:160, HLA-A02:162, HLA-A02:163, HLA-A02:164, HLA-A02:165, HLA-A02:166, HLA-A02:168, HLA-A02:251, HLA-A02:252, HLA-A02:256, HLA-A02:257, HLA-A02:145, HLA-A02:149, HLA-A02:150, HLA-A02:192, HLA-A02:193, HLA-A02:194, HLA-A02:196, HLA-A02:197, HLA-A02:198, HLA-A02:199, HLA-A02:200, HLA-A02:201, HLA-A02:228, HLA-A02:234, HLA-A02:235, HLA-A02:236, HLA-A02:260, HLA-A02:266, HLA-A02:182, HLA-A02:183, HLA-A02:185, HLA-A02:187, HLA-A02:189, HLA-A02:190, HLA-A02:121, HLA-A02:123, HLA-A02:161, HLA-A02:35, HLA-A02:38, HLA-A02:139, HLA-A02:262, HLA-A02:41, HLA-A02:58, HLA-A02:233, HLA-A02:147, HLA-A02:151, HLA-A02:167, HLA-A02:20, HLA-A02:122, HLA-A02:44, HLA-A02:142, HLA-A02:34, HLA-A02:42, HLA-A02:78, HLA-A02:06, HLA-A02:21, HLA-A02:28, HLA-A02:51, HLA-A02:61, HLA-A02:72, HLA-A02:79, HLA-A02:91, HLA-A02:106, HLA-A02:180, HLA-A02:137, HLA-A02:170, HLA-A02:248, HLA-A02:144, HLA-A02:259, HLA-A02:126, HLA-A02:243, HLA-A02:52, HLA-A02:48, HLA-A02:60, HLA-A02:62, HLA-A02:127, or HLA-A02:229 molecule; or
the missense mutation at position 12 of the KRAS protein is G12C, and wherein the HLA-A*02 molecule is an HLA-A02:253, HLA-A02:03, HLA-A02:264, HLA-A02:258, HLA-A02:230, HLA-A02:69, HLA-A02:11, HLA-A02:104, HLA-A02:22, HLA-A02:50, HLA-A02:128, HLA-A02:26, HLA-A02:171, HLA-A02:99, HLA-A02:102, HLA-A02:155, HLA-A02:63, HLA-A02:02, HLA-A02:186, HLA-A02:115, HLA-A02:209, HLA-A02:47, HLA-A02:13, HLA-A02:141, HLA-A02:90, HLA-A02:148, HLA-A02:158, HLA-A02:131, HLA-A02:16, HLA-A02:263, HLA-A02:116, HLA-A02:29, HLA-A02:35, HLA-A02:38, HLA-A02:105, HLA-A02:12, HLA-A02:245, HLA-A02:73, HLA-A02:241, HLA-A02:71, HLA-A02:59, HLA-A02:40, HLA-A02:166, HLA-A02:238, HLA-A02:176, HLA-A02:75, HLA-A02:30, HLA-A02:174, HLA-A02:266, HLA-A02:187, HLA-A02:85, HLA-A02:165, HLA-A02:160, HLA-A02:183, HLA-A02:189, HLA-A02:138, HLA-A02:228, HLA-A02:260, HLA-A02:107, HLA-A02:215, HLA-A02:182, HLA-A02:09, HLA-A02:192, HLA-A02:163, HLA-A02:221, HLA-A02:159, HLA-A02:194, HLA-A02:140, HLA-A02:206, HLA-A02:74, HLA-A02:198, HLA-A02:123, HLA-A02:95, HLA-A02:168, HLA-A02:150, HLA-A02:210, HLA-A02:86, HLA-A02:235, HLA-A02:237, HLA-A02:208, HLA-A02:212, HLA-A02:201, HLA-A02:120, HLA-A02:240, HLA-A02:211, HLA-A02:175, HLA-A02:162, HLA-A02:121, HLA-A02:89, HLA-A02:220, HLA-A02:164, HLA-A02:190, HLA-A02:157, HLA-A02:96, HLA-A02:256, HLA-A02:234, HLA-A02:97, HLA-A02:204, HLA-A02:70, HLA-A02:77, HLA-A02:93, HLA-A02:181, HLA-A02:111, HLA-A02:118, HLA-A02:196, HLA-A02:185, HLA-A02:214, HLA-A02:193, HLA-A02:200, HLA-A02:25, HLA-A02:173, HLA-A02:177, HLA-A02:207, HLA-A02:257, HLA-A02:203, HLA-A02:199, HLA-A02:66, HLA-A02:01, HLA-A02:216, HLA-A02:133, HLA-A02:119, HLA-A02:153, HLA-A02:251, HLA-A02:145, HLA-A02:24, HLA-A02:197, HLA-A02:236, HLA-A02:149, HLA-A02:68, HLA-A02:218, HLA-A02:205, HLA-A02:31, HLA-A02:239, HLA-A02:109, HLA-A02:67, HLA-A02:132, HLA-A02:134, HLA-A02:252, HLA-A02:202, HLA-A02:213, HLA-A02:161, HLA-A02:122, HLA-A02:27, HLA-A02:262, HLA-A02:233, HLA-A02:41, HLA-A02:139, HLA-A02:44, HLA-A02:142, HLA-A02:58, HLA-A02:229, HLA-A02:167, HLA-A02:147, or HLA-A02:151 molecule.

5. (canceled)

6. (canceled)

7. An antigen targeting agent as defined in claim 1, the agent comprising first and second chains, each one of the first and second chains having first, second and third complementarity determining regions (CDRs), wherein:
the third CDR of the first chain comprises the amino acid sequence of SEQ ID NO:30 or SEQ ID NO:34, and wherein the third CDR of the second chain comprises the amino acid sequence of SEQ ID NO:32 or SEQ ID NO:36;
the first chain comprises the amino acid sequence of TRAV27*01 (SEQ ID NO:6) or the amino acid sequence of TRAV13-2*01 (SEQ ID NO:10);
the second chain comprises the amino acid sequence of TRBV 19*01 (SEQ ID NO:8) or the amino acid sequence of TRBV 04-1*01 (SEQ ID NO:12);
the first CDR of the first chain comprises SEQ ID NO:14 or SEQ ID NO:18;
the second CDR of the first chain comprises SEQ ID NO:16 or SEQ ID NO:20;
the first CDR of the second chain comprises SEQ ID NO:22 or SEQ ID NO:26;
the first CDR of the second chain comprises SEQ ID NO:22 or SEQ ID NO:26; and/or
the second CDR of the second chain comprises SEQ ID NO:24 or SEQ ID NO:28.

8. (canceled)

9. (canceled)

10. (canceled)

11. (canceled)

12. (canceled)

13. (canceled)

14. An antigen targeting agent as defined in claim 1, wherein:
the first chain comprises as its first, second and third CDRs SEQ ID NO:14, SEQ ID NO:16 and SEQ ID NO:30, respectively, and the second chain comprises as its first, second and third CDRs SEQ ID NO:22, SEQ ID NO:26 and SEQ ID NO:32, respectively;
the first chain comprises as its first, second and third CDRs SEQ ID NO:18, SEQ ID NO:20 and SEQ ID NO:34, respectively, and the second chain comprises as its first, second and third CDRs SEQ ID NO:22, SEQ ID NO:24 and SEQ ID NO:32, respectively;
the first chain comprises as its first, second and third CDRs SEQ ID NO:14, SEQ ID NO:16, and SEQ ID NO:30, respectively, and the second chain comprises as its first, second and third CDRs SEQ ID NO:26, SEQ ID NO:28 and SEQ ID NO:36, respectively; or
the first chain comprises as its first, second and third CDRs SEQ ID NO:18, SEQ ID NO:20 and SEQ ID NO:34, respectively, and the second chain comprises as its first, second and third CDRs SEQ ID NO:26, SEQ ID NO:28 and SEQ ID NO:36, respectively.

15. An antigen targeting agent as defined in claim 1, wherein:
the missense mutation at position 12 of the KRAS is G12V, and the third CDR of the first chain has the amino acid sequence of SEQ ID NO:30 and the third CDR of the second chain has the amino acid sequence of SEQ ID NO:32;
the missense mutation at position 12 of the KRAS is G12D, and the third CDR of the first chain has the amino acid sequence of SEQ ID NO:34 and the third CDR of the second chain has the amino acid sequence of SEQ ID NO:32; or
the missense mutation at position 12 of the KRAS is G12D, and the third CDR of the first chain has the amino acid sequence of SEQ ID NO:30 and the third CDR of the second chain has the amino acid sequence of SEQ ID NO:36.

16. An antigen targeting agent as defined in claim 1, wherein:
the first and second chains of the antigen targeting agent comprise a single polypeptide, or wherein the first and second chains of the antigen targeting agent comprise two separate polypeptides;
the first and second chains of the antigen targeting agent are configured to be expressed as a single polypeptide with a suitable sequence interposing the first and second chains so that the first and second chains are cleaved into or translated as two separate polypeptides in vivo, wherein the suitable sequence optionally comprises a T2A, P2A, E2A, F2A or IRES sequence.

17. (canceled)

18. An antigen targeting agent as defined in claim 1, wherein the antigen targeting agent comprises a T-cell receptor (TCR), wherein optionally:
the first chain comprises an alpha-chain of the TCR, and the second chain comprises a beta-chain of the TCR;
the first chain comprises a gamma-chain of the TCR, and the second chain comprises a delta-chain of the TCR;
constant regions of the TCR comprise murine constant regions; and/or
the T-cell receptor comprises the amino acid sequence of any one of SEQ ID NOs:38, 40, 42 or 44.

19. (canceled)

20. (canceled)

21. (canceled)

22. An antigen targeting agent as defined in claim 1, wherein the antigen targeting agent comprises a chimeric antigen receptor (CAR), and wherein the three complementarity determining regions of each of the first and second chains are configured to be expressed as a single polypeptide together with a co-stimulatory domain; or wherein the antigen targeting agent comprises a bi-specific antibody, the bi-specific antibody having a first domain comprising the antigen-binding site that binds to the KRAS protein having a missense mutation at position 12 when the peptide incorporating the missense mutation is presented by an HLA-A*02 molecule, and a second domain comprising an antigen binding site configured to recruit cytotoxic cells, optionally wherein the second domain of the bi-specific antibody binds CD3.

23. (canceled)

24. (canceled)

25. An antigen targeting agent as defined in claim 1, wherein the antigen targeting agent specifically binds to the peptide incorporating the missense mutation at position 12 of the KRAS protein when the peptide is presented by an HLA-A*02 molecule; or wherein the antigen targeting agent is expressed by a cell that has been genetically engineered to express the antigen targeting agent.

26. (canceled)

27. (canceled)

28. An isolated or purified antigen targeting agent as defined in claim 1.

29. An isolated nucleic acid molecule having a DNA sequence encoding an antigen targeting agent as defined in claim 1.

30. An isolated nucleic acid molecule as defined in claim 29 having the nucleotide sequence of any one of SEQ ID NOs:37, 39, 41, 43, 45, 46, 47 or 48.

31. A pharmaceutical composition comprising an antigen targeting agent as defined in claim 1 and a pharmaceutically acceptable carrier.

32. A cytotoxic cell that has been genetically engineered to express an antigen targeting agent as defined in claim 1.

33. A cytotoxic cell comprising a nucleic acid molecule as defined in claim 30.

34. A cytotoxic cell as defined in claim 32, wherein the cytotoxic cell is a CD8⁺ T-cell, CD4⁺ T-cell or natural killer cell.

35. A method of producing a cytotoxic cell capable of expressing an antigen targeting agent to bind KRAS peptides having a missense mutation at position 12 as presented by HLA-A*02 molecules, the method comprising:
obtaining cytotoxic cells from a source; and
genetically engineering the cytotoxic cells using a nucleotide vector comprising the nucleic acid molecule of claim 29.

36. (canceled)

37. (canceled)

38. (canceled)

39. (canceled)

40. (canceled)

41. (canceled)

42. (canceled)

43. (canceled)

44. A method of detection of cancer in a mammalian subject, the method comprising:
contacting a sample comprising cells obtained from the subject with an antigen targeting agent or a cytotoxic cell as defined in claim 1;
if the cells express KRAS^{G12X} antigens, the antigen targeting agent or the cytotoxic cell binds to the KRAS^{G12X} antigens, thereby forming a complex; and
detecting the presence of the complex, wherein the presence of the complex is indicative of cancer in the mammal; or the method comprising:
obtaining a sample from the subject;
co-culturing cells from the sample with cytotoxic cells capable of binding to KRAS^{G12X} peptides as displayed by HLA-A*02 molecules, wherein the cytotoxic cells express an antigen targeting agent as defined in claim 1; and
evaluating an indicator of cytotoxic activity;
wherein a presence of or increase in a level of the indicator of cytotoxic activity indicates a cancer involving a missense mutation at position 12 of KRAS.

45. (canceled)

46. (canceled)

47. (canceled)

48. (canceled)

49. (canceled)
